(19) United States
(12) Patent Application Publication
(10) Pub. No.: US 2026/0125779 A1
(43) Pub. Date: May 7, 2026

(54) ALLOYS FOR IMPLANTS

(71) Applicant: Washington State University, Pullman, WA (US)

(72) Inventors: Amit Bandyopadhyay, Pullman, WA (US); Susmita Bose, Pullman, WA (US); Sushant R. Ciliveri, Pullman, WA (US); Indranath Mitra, Williamsburg, VA (US); William S. Dernell, Potlatch, ID (US); Jose D. Avila, Toppenish, WA (US)

(73) Assignee: Washington State University, Pullman, WA (US)

(21) Appl. No.: 19/354,120

(22) Filed: Oct. 9, 2025

Related U.S. Application Data
(60) Provisional application No. 63/705,984, filed on Oct. 10, 2024.

(51) Int. Cl. C22C 14/00; A61L 27/06; B33Y 10/00; B33Y 80/00

(52) U.S. Cl. CPC C22C 14/00; A61L 27/06; B33Y 10/00; B33Y 80/00

(57) ABSTRACT

A biocompatible alloy that includes titanium, copper, and at least one metallic element selected from: tantalum, niobium, zirconium, zinc, tungsten, lithium, potassium, strontium, sodium, calcium, chromium, molybdenum, tin, manganese, iron, and cobalt.

This application claims the benefit of U.S. Provisional Appl. No. 63/705,984, filed Oct. 10, 2024, which is incorporated herein by reference in its entirety.

### ACKNOWLEDGMENT OF GOVERNMENT SUPPORT

This invention was made with government support under grant numbers R01 AR067306 and R01 AR078241 awarded by the National Institutes of Health. The government has certain rights in the invention.

### BACKGROUND

Early-stage osseointegration is one of the most desirable qualities of metallic implants because it ensures faster healing and long-term implant stability, primarily depending on the implant's biocompatibility. Compromised biocompatibility due to bacterial growth on the implant has been shown to result in adverse events like septic loosening and prosthetic joint infection (PJI), which ultimately requires revision surgeries to mitigate such clinical challenges.

Growth in the incidence of revision surgeries due to PJI is projected to be 176% and 170% for THA and TKA by 2030, supporting prior literature suggesting a losing battle against this postoperative outcome. A recent World Health Organization report states that around 700,000 deaths occur yearly due to antimicrobial resistance (AMR). If no clinical remedies are found, as many as 10 million deaths per year are predicted by 2050 due to infection, higher than 8.2 million deaths per year due to cancer and will become a significant economic burden worldwide. In addition, the mortality rate for PJI is 87.3%, which is greater than those for colorectal and lung cancer and comparable to those for breast cancer (89%), which makes PJI a compelling and critical clinical challenge that needs immediate attention. Available therapy is based on a two-pronged approach of 1) extensive local debridement and implant replacement via revision surgery and 2) antibiotic treatments at the local surgery site or through systemic administration. However, those approaches are not always sustainable, leading to recurring infections even after revision surgery.

The interdependence of high infection rate (postoperative spine infection 0-18% and knee/hip arthroplasty 1-2%, revision surgeries (8-15% for arthroplasty), and out-of-pocket costs associated with such procedures (up to $93,000 in 2009) further complicate the problem. The most common infections originate from either Staphylococcus aureus (S. aureus˜66%) or Pseudomonas aeruginosa (P. aeruginosa˜15%, causing a recurrent infection rate for S. aureus following revision surgeries as high as 75% while only 56% are cured at 1-year post-op. In addition, infection of diverse types of implants such as hip, knee, and spine need highly heterogeneous modes of treatment, which can make healthcare complicated and expensive. This heterogeneity heavily biases material evaluation such as in vivo failure analyses, biological response, and implant success as a function of material properties. Therefore, implants should be self-sufficient to prevent prosthetic joint infections for better material evaluation and to mitigate the complexities of revision surgeries. Multifunctional materials such as Ti—Cu alloys can ensure implant success, access to healthcare use-costs, and increase value-of-product. So far, most studies have evaluated Ti implants alloyed with ≥5% Cu for bacterial resistance fabricated via powder metallurgy. However, the general perspective on such high amounts of copper addition in implant materials is linked to scientific concerns. Moreover, these studies do not account for any cytotoxicity from higher amounts of copper or evaluate ways of comprehensively improving the osseointegration ability of the implants.

Accordingly, a need exists for new methods and compositions for fortifying implants, in particular, titanium implants, to inherently protect the implant against bacterial infection while enhancing biocompatibility. In particular, alloying with, for example, metallic copper in a range from about 0.01 to about 30 wt %, based on the total weight of the alloy, will make the implant compositions disclosed herein bacteria-resistant long-term, leading to enhanced early-stage osseointegration and aids in resisting bacterial invasion and enhances osseointegration for faster healing and bone regeneration.

### SUMMARY

Disclosed herein is a biocompatible alloy comprising:
titanium, copper, and
at least one metallic element selected from: tantalum, niobium, zirconium, zinc, tungsten, lithium, potassium, strontium, sodium, calcium, chromium, molybdenum, tin, manganese, iron, and cobalt.

Also disclosed herein is a biocompatible alloy comprising (i) titanium, (ii) copper and (iii) tantalum, niobium, or a mixture thereof.

Further disclosed herein is a biocompatible alloy comprising (i) titanium, (ii) copper, and (iii) magnesium oxide, silicon dioxide, or a mixture thereof.

The foregoing will become more apparent from the following detailed description, which proceeds with reference to the accompanying figures.

### BRIEF DESCRIPTION OF THE DRAWINGS

FIG. 1A shows an example schematic of synergistic antibacterial efficacy and enhanced biocompatibility of Ti3Al2V—Cu—Ta alloys toward early-stage osseointegration.

FIG. 1B shows histology micrographs of tissue cross-sections at the bone-implant interface.

FIG. 2A shows a microstructure of samples cut along the build direction showing acicular α′ martensitic needles Ti3Al2V, Ti3Al2V-2Cu, and typical α and β microstructures for (Commercially pure titanium) CpTi and Ti6Al4V, respectively. Note that the needle-like acicular structure shifts to a smoother lamellar microstructure for Ti3Al2V-3Cu, Ti3Al2V-10Ta and Ti3Al2V-10Ta-3Cu compositions.

FIG. 2B shows phase analysis from X-ray diffraction; peak shifts observed for Ti6Al4V and Ti3Al2V compared to (Commercially pure titanium) CpTi due to the addition of alloying elements Al and V. Higher peak shift observed for Ti6Al4V compared to Ti3Al2V. The intensity of all α-Ti peaks except (002) was highest for CpTi due to the introduction of β phase stabilizer V in Ti6Al4V and Ti3Al2V, resulting in reduced amounts of α-Ti. Peak intensity for (002) α-Ti, however, was highest for Ti6Al4V, followed by Ti3Al2V, and lowest for (Commercially pure titanium) CpTi since this coincides with an enhanced stabilized (011) β-Ti peak. A right-shifted peak for Ti3Al2V-3Cu and a left-shifted peak for Ti3Al2V-10Ta-3Cu were observed due to Cu acting as a β-Ti stabilizer, while Ta addition results in supersaturated bcc Ti—Ta solid solution, respectively.

FIG. 2C shows SEM of fracture surface at the porous-dense interface from the shear test for Ti6Al4V-D, Ti6Al4V-P20, Ti6Al4V-P40, Ti3Al2V-D, Ti3Al2V-P20, and Ti3Al2V-P40. Dimple features were observed at the fracture interface for all compositions, indicating ductile fracture; dimples were more prominent in Ti3Al2V compared to Ti6Al4V, indicating a desired higher ductile behavior for Ti3Al2V before failure.

FIG. 2D shows elastic modulus and compressive yield strength evaluated from the raw compressive stress-strain data plotted against their measured porosities. The effect of reduced Al and V in Ti6Al4V, i.e., Ti3Al2V, showed no prominent effect in elastic modulus compared to Ti6Al4V. However, compressive yield strength decreased for Ti3Al2V for both dense and porous structures. Adding 3% Cu increased the compressive yield strength compared to Ti3Al2V-2Cu; however, no strength increase is seen due to 10% Ta addition. Shear modulus and maximum shear strength were evaluated by shearing the structures at the porous-dense interface using a single-shear test device plotted against their measured porosities. Reduced amounts of Al and V in Ti6Al4V, i.e., Ti3Al2V, had a noticeable reduction in both the shear modulus and the maximum shear strength endured at the porous-dense interface.

FIG. 3A shows an XRD plot for all compositions showing alterations in diffraction patterns between different alloys.

FIG. 3B shows Vickers microhardness values for Ti6Al4V, Ti3Al2V, Ti3Al2V-2Cu, Ti3Al2V-3Cu, and Ti3Al2V-10Ta-3Cu compositions.

FIG. 4A Shear stress-strain plot from raw data for dense and porous Ti6Al4V and Ti3Al2V. Lower shear modulus and shear strengths were observed for Ti3Al2V compared to Ti6Al4V. Higher ductility behavior prior to failure was observed for Ti3Al2V-D and Ti3Al2V-P20 compared to Ti6Al4V-D and Ti6Al4V-P20, respectively. However, similar ductility prior to failure was observed for Ti3Al2V-P40 and Ti6Al4V-P40. Compressive stress-strain plot from raw data for dense and porous Ti6Al4V, Ti3Al2V, Ti3Al2V-3Cu, and Ti3Al2V-10Ta-3Cu.

FIG. 4B-4D show compressive strain plots from raw data for dense and porous Ti6Al4V, Ti3Al2V, Ti3Al2V-3Cu, and Ti3Al2V-10Ta-3Cu: FIG. 4B dense (−D), FIG. 4C 20% porosity (−P20), and FIG. 4D 40% porosity (−P40) structures.

FIG. 5A shows pictograms of the wear track and wear ball for CpTi, Ti6Al4V, Ti3Al2V, Ti3Al2V-10Ta, Ti3Al2V-3Cu, and Ti3Al2V-10Ta-3Cu. Scale bar represents 500 μm and is uniform or all wear track images.

FIG. 5B shows tribology testing displaying the wear track's formation.

FIG. 5C shows measured wear track width. Statistical analyses were done using one-way ANOVA for a=0.05. Tukey-Kramer simulations were done for pairwise comparisons of wear track width with a P-value<0.05 considered significantly different.

FIG. 5D shows during Tribological testing, acquisition of the compound wear.

FIG. 5E shows during Tribological testing, coefficient of friction (COF).

FIG. 5F shows during Tribological testing, OCP measurement curves.

FIG. 5G also shows during Tribological testing, OCP measurement curves.

FIG. 6A shows a schematic of in vivo native bone integration into surgically placed metallic implants showing a variation of osseointegration ability as a function of material properties.

FIG. 6B shows histology micrographs of tissue cross-sections at the bone-implant interface stained with Hematoxylin and Eosin stain (H&E) for porous implants. The black area is the implant (denoted with triangles), with a dark area representing the trabecular bone formed (square), lighter areas with a smooth appearance is fibrocartilage, the light area with rounded cell nuclei is osteoid tissue, and dots in osteoid tissue represents the osteoblast cells in the bone integration front (circle). The pore cross-section shown is not the same for all compositions. Infiltration of newly formed trabecular bone into the pores is observed for Ti3Al2V-P along with good amounts of matured bone on the implant's outer surface compared to CpTi (gaps at the BIC) and Ti6Al4V. No evidence of inflammatory response was noticed for any composition. The scale bar in the first image represents 25 μm and is uniform for all images.

FIG. 7 shows Sanderson's Rapid Bone-Stained micrographs (SRBS). Areas (shown as Reddish/orange) represent matured trabecular bone, while darker and lighter (shown as blue) areas represent osteoid tissue or a combination of fibrocartilage with osteoid tissue, respectively. Dark ((shown as blue rounded dots) are osteoblasts recruited in the newly formed osteoid tissue at the bone integration front. Some areas of a (shown as lighter blue) with more elongated cells represent chondrocyte cells in the fibrocartilage. Ti3Al2V clearly shows bone apposition and osseointegration at the BIC compared to clear gaps for CpTi and Ti6Al4V. The presence of well-embedded osteocytes in the trabecular area for Ti6Al4V composition at 4 weeks post-implantation suggests older bone that could be a residual continuation from the outer cortical area. Adding Cu to Ti3A13V-3Cu composition does not elicit any inflammation or neoplasia, suggesting non-toxicity; however, there is evidence of the delayed onset of osseointegration compared to Ti3Al2V composition alone. Positive control Ti3Al2V-10Ta shows very well-integrated trabecular bone at the BIC with trabecular bone width higher than the rest of the compositions. Interestingly, adding 10% Ta to Ti3A13V-3Cu reverses the delayed onset of osseointegration and shows the overall best performance among all six compositions. Graphs present a quantitative analysis of the observations made from SRBS-stained histology sections based on ImageJ Trainable Weka Segmentation. The scale bar in the first image represents 25 μm and is uniform for all images.

FIG. 8 shows histology micrographs of tissue cross-sections at the bone-implant interface stained with Sanderson's Rapid Bone Staining (SRBS). The black area is the implant with the area (shown as reddish) representing the trabecular bone formed, bluish area as the osteoid tissue and blue dots in white area represents the osteoblast cells in the bone marrow. Bone areas (shown as reddish orange) with black crack-like features represents compact bone present pre-implantation. Newly grown trabecular bone is directly apposed to the implant surface in CpTi-D and Ti3Al2V-D. Osteoid lining can be seen to be apposed to the implant surface followed by trabecular bone areas in Ti6Al4V-D, indicating slower early-stage osseointegration in comparison to CpTi-D and Ti3Al2V-D.

FIG. 9 shows histology micrographs of tissue cross-sections at the bone-implant interface stained with Hematoxylin and Eosin (H&E) staining with the black area as the implant, the formed trabecular bone (shown as a deep purple color) and the osteoblast cells in the bone marrow (as shown as the purple and orange dots in white region). No evidence of inflammatory response was noticed.

FIG. 10A shows a schematic of intrinsically antibacterial material intervention towards post-surgical secondary infection at the surgery site. Bacterial inhibition characteristics of a material depend on the bacterial type, cell wall structure, and mode of killing.

FIG. 10B shows Pseudomonas aeruginosa bacterial study after 36 hrs of culture; cetrimide agar plate images showing bacterial colonies across all compositions; SEM images showing the deflated bacterial cell wall-morphology for Ti3Al2V-2Cu and Ti3Al2V-3Cu due to cytoplasmic outflow as a result of toxicity from the Cu towards bacterial cells.

FIG. 10C shows bacterial cell viability and bacterial colony count from agar plate counting (n=3), showing decreased bacterial viability for Ti3Al2V, Ti3Al2V-2Cu, and Ti3Al2V-3Cu compared to CpTi and Ti6Al4V.

FIG. 10D shows Staphylococcus aureus bacterial cell culture; bacterial cell viability and bacterial cell count from SEM images (n=4) showing decreased bacterial viability for Ti3Al2V, Ti3Al2V-2Cu, and Ti3Al2V-3Cu compared to CpTi and Ti6Al4V with cytoplasmic outflow observed even as early as 24 h.

FIG. 11. Schematic of MgO-induced osteogenic activity towards early stage osseointegration and bactericidal effect of Cu in CpTi. The CpTi—MgO—Cu was processed via metal-AM, enabling the incorporation of designed porosity. This further expedites the bone-remodeling and tissue attachment on the implant's surface, contributing to its long-term stability in vivo.

FIG. 12. (a) Image of the build plate after DED operation showing dense discs printed on a CpTi build plate and a schematic of the top surface of the discs used for microstructure and microhardness evaluations. (b) Microstructure of polished samples perpendicular to the build direction processed via DED-based AM technique. CpTi and CpTi—MgO show α′ martensitic acicular needle structure typically observed in AM-processed CpTi due to the fast cooling nature of the process. CpTi—MgO—Cu shows keyhole porosities on the surface due to balling effect and material splashing in the melt-pool owing to high thermal diffusivity and low laser absorption demonstrated by Cu. (c) Vickers microhardness (HV0.2) evaluation for the compositions reveal enhancement in hardness with MgO addition in CpTi due to ceramic reinforcement. Hardness further increased with Cu addition in CpTi—MgOdue to Ti2Cu intermetallic formation. Hardness values were subjected to a one-way ANOVA test for n=5 and α<0.05. Tukey-Kramer correction simulation was carried out for pairwise comparison of means with a P value<0.05 considered significantly different and marked with an ‘*’. Hardness values for all compositions were found to be statistically different from each other.

FIG. 13. Histology images of in vivo bone sections using a rat distal femur model after 6 weeks of surgical implantation. A feature legend for each stain representing particular features is presented. The implant area has been denoted with triangles in each histology image. (a) Gomori trichrome stain represents the growth of muscle fibers and collagen formation at the bone-implant contact (BIC). Visible gaps were observed at the BIC for CpTi, indicating poor osseointegration performance. Similar gaps at the BIC for CpTi—MgO are filled with mineralization fronts. Muscle fiber interwoven with collagen formation apposed directly to the implant surface was observed for CpTi—MgO and CpTi—MgO—Cu. (b) Hematoxylin and Eosin (H&E) stain showing varying shades of pink representing bone-remodeling features. No inflammatory markers indicating necrosis or neoplasia were observed at the BIC. Trabecular bone formation apposed to the implant surface was observed for CpTi—MgO with good pore infiltration. CpTi—MgO—Cu shows a lower degree of trabecular bone at the BIC than CpTi—MgO, indicating delayed osseointegration owing to Cu presence. (c) Sanderson's Rapid Bone Stained (SRBS) bone sections indicate mineralized bone with well-embedded osteocytes apposed directly to the implant surface with infiltration into the implant region for CpTi—MgO. In contrast, CpTi shows an osteoid lining at the BIC, indicating delayed osseointegration compared to CpTi—MgO. Quantitative analysis of mineralized bone formation at the BIC represents four-fold bone formation for CpTi—MgO than CpTi. CpTi—MgO—Cu shows lower amounts of trabecular bone formation than CpTi—MgO, eliciting delayed osseointegration. Statistical analysis was done with one-way ANOVA for mineralized bone formation for n≥7 and α<0.05. Tukey-Kramer simulations were conducted for pairwise comparison with a P value<0.05 considered significantly different. Mineralized bone formation for CpTi—MgO and CpTi—MgO—Cu were statistically similar and higher than that for CpTi.

FIG. 14. Staphylococcus aureus bacterial study after 24, 48, and 72 hrs of culture; (a) agar plate images after 24 hrs, and SEM images at 3000× magnification after 24, 48, and 72 hrs of culture. Respective bacterial colony counts and corresponding % bacterial viability to CpTi as the negative control have been presented. Reduction in planktonic bacteria on the CpTi-MgO—Cu surface can be significantly lower than CpTi at all time points due to the bactericidal effect demonstrated by Cu presence. SEM counting (n=4) shows a gradual reduction in % bacterial viability on CpTi—MgO—Cu from 24 to 72 hrs, with the highest antibacterial efficacy at 72 hrs. (b) High magnification SEM images showing the on-contact bacterial killing of S. aureus bacterial cells with deflated morphology, ruptured cell membrane, and cytoplasm outflow post 48 and 72 h of culture.

FIG. 15. A schematic of the synergistic effect of SiO₂ and Cu presence in CpTi towards enhanced early-stage bone tissue growth, integration with host-bone, and antibacterial performance, respectively. The figure displays low load-bearing dental implants and cranial prosthesis and porous coating on high-strength Ti6Al4V as high load-bearing implants. Fabrication of these implants via AM enables the introduction of designed porosities that aid the osseointegration process.

FIG. 16. (a) Microstructures of polished and etched surfaces for DED-printed CpTi, CpTi-SiO₂, and CpTi-SiO₂-3Cu. Martensitic α′ needle-like structures are seen for CpTi and CpTi-SiO₂ compositions typically observed in additively manufactured CpTi. CpTi-SiO₂-3Cu shows keyhole porosities on the surface due to the balling effect exhibited by Cu's high thermal diffusivity. (b) Vickers microhardness values (HV0.2) reveal hardness enhancement in CpTi-SiO₂ due to SiO₂ ceramic incorporation and further hardness enhancement for CpTi-SiO₂-3Cu due to Ti2Cu intermetallic phase formation. Hardness values were subjected to a one-way ANOVA test for n=5 and α<0.05. Tukey-Kramer simulations were done for pairwise comparisons; a P value<0.05 was marked as a significant difference with an ‘*’. (c) Phase analysis using XRD revealed peaks observed for α′—Ti phase. No individual peaks for SiO₂ or Cu were observed, indicating a homogeneous distribution.

FIG. 17. (a) Schematic of the location of the implant in the lateral epicondyle region of the rat's distal femur in vivo and the progression of early-stage osseointegration via tissue growth and attachment on the implant's surface with host-bone, with osteoblast recruitment followed by matured bone formation over 6 weeks. (b) Gomori trichrome stained in vivo bone section micrographs reveal gaps at the bone-implant interface for CpTi, indicating poor osseointegration. CpTi-SiO₂ and CpTi-SiO₂—Cu compositions display no gaps at the interface. On the contrary, the mineralization fronts are observed to be infiltrated into the implant region with collagen presence interwoven into the muscle fibers. (b) Hematoxylin and Eosin (H&E) stained bone section micrographs show no markers of inflammation. Mineralized bone formation at the bone-implant contact is absent in some areas for CpTi. CpTi-SiO₂ and CpTi-SiO₂-3Cu show mineralized bone directly apposed to the implant's outer surface. The implant regions in each image are black and represented with a triangle. Legend bars representing distinct features for each staining are provided above the micrographs. CpTi-SiO₂ and CpTi-SiO₂-3Cu show superior early-stage osseointegration performance compared to CpTi.

FIG. 18. Sanderson's Rapid Bone Stained (SRBS) in vivo bone section micrographs display distinct features of osteoid lining, osteoblast recruitment, and mineralized bone formation. Mineralized bone formation was absent in some regions at the bone-implant contact for CpTi. CpTi—SiO₂ and CpTi—SiO₂-3Cu show thicker regions of bone formation at the interface. Tissue infiltration inside the pores for CpTi—SiO₂-3Cu shows an absence of matured bone due to delayed osseointegration. Quantitative histomorphometry for mineralized bone formation at the bone-implant contact reveals the highest bone formation for CpTi—SiO₂, followed by CpTi—SiO₂-3Cu, and lowest for CpTi. The mineralized bone formation was evaluated as % area from at least n=7 distinct regions around the bone-implant interface and subjected to a one-way ANOVA test with α<0.05. Tukey Kramer simulations for pairwise comparisons with α<0.05 as statistically different and marked with an ‘*’ reveal statistical differences for CpTi and CpTi—SiO₂/CpTi—SiO₂-3Cu. CpTi—SiO₂ and CpTi—SiO₂-3Cu showed statistically similar bone formation at the bone-implant contact.

FIG. 19. (a) Agar plate images after 24 h, and SEM images (5000× magnification) after 24-48-72 h of the culture of S. aureus bacteria on CpTi and CpTi—SiO₂-3Cu. The respective bacterial colony counts (n=3 for agar plate, n=4 for SEM images) and % bacterial viability to CpTi are shown in the plots. After 24 h, the number of bacterial colonies on the agar plate for CpTi—SiO₂—Cu can be observed compared to CpTi due to the antibacterial action of Cu. SEM images show a reduction in planktonic S. aureus bacteria on the CpTi—SiO₂-3Cu surface compared to CpTi. Bacterial counts show a gradual reduction in bacterial viability on CpTi-SiO₂-3Cu, with the least % bacterial viability (highest antibacterial efficiency) at the end of 72 h. (b) SEM images at higher magnification reveal the on-contact killing mechanism of Cu, with ruptured bacterial cell membrane walls (48 h), cytoplasm outflow, and deflated bacterial cell morphology (72 h).

### DETAILED DESCRIPTION

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

“Ti6Al4V” designates a titanium alloy with 6 wt. % Al and 4 wt. % V, based on the total weight of the alloy.

“Ti3Al2V” designates a titanium alloy with 3 wt. % Al and 2 wt. % V, based on the total weight of the alloy.

Disclosed herein are biological implant materials, particularly bone implant materials. In one aspect, the bone implant materials are additively manufactured compositions for load-bearing implants.

An aspect of the disclosure herein demonstrates that alloys, such as, but not limited to, Ti—Ta—Cu, provided inherent bacterial resistance suitable for load-bearing applications. Instead of using Ti6Al4V alloy, vanadium and aluminum contents were configured to design a Ti3Al2V alloy for metallic implant applications. Ta (e.g., 10 wt %) and Cu (e.g., 3 wt %) were added to the Ti3Al2V alloy to enhance biocompatibility and impart inherent bacterial resistance. Additively manufactured implants were thus utilized for resistance against Pseudomonas aeruginosa and Staphylococcus aureus strains of bacteria up to 72 h. The Cu addition to Ti3Al2V showed a surprising and unexpectedly improved antibacterial efficacy, i.e., 80% higher than CpTi (Commercially Pure Titanium) and Ti6Al4V. Resultant mechanical properties for Ti3Al2V-10Ta-3Cu demonstrated excellent fatigue resistance, good shear strengths, and better tribological characteristics than Ti6Al4V. In vivo studies using a rat distal femur model showed improved early-stage osseointegration for alloys with the, for example 10% Ta addition compared to CpTi and Ti6Al4V. The results show that a beneficial Ti3Al2V-10Ta-3Cu alloy synergistically improves in vivo biocompatibility and induces the inherent ability toward microbial resistance for the next generation of load-bearing metallic implants.

In certain embodiments, the alloy includes (i) titanium, (ii) copper, and (iii) tantalum and/or niobium. The tantalum and/or niobium improves biocompatibility, and the copper provides antibacterial resistance. In certain embodiments, the alloy also includes aluminum and vanadium. In certain embodiments, the alloy also includes zinc. Particular alloys include Ti—Ta—Cu; Ti—Ta—Cu—Al—V; Ti—Nb—Cu; Ti—Nb—Cu—Al—V; Ti—Ta—Nb—Cu; Ti—Ta—Nb—Cu—Zn; and Ti—Ta—Cu—Zn.

In certain embodiments, the copper is present in the alloy in an amount of about 0.01 wt % to about 30 wt %, more particularly about 0.01 wt % to about 15 wt %. In certain embodiments, the tantalum is present in the alloy in an amount of about 0.01 wt % to about 50 wt %. In certain embodiments, the niobium is present in the alloy in an amount of about 0.01 wt % to about 50 wt %. In certain embodiments, the aluminum is present in the alloy in an amount of about 0.01 wt % to about 10 wt %. In certain embodiments, the vanadium is present in the alloy in an amount of about 0.01 wt % to about 10 wt %. In certain embodiments, the zincs present in the alloy in an amount of about 0.01 wt % to about 10 wt %. In certain embodiments, titanium constitutes the remaining amount of the alloy.

In certain embodiments the alloy is manufactured using processes such as forging or casting, or is manufactured additively using powder sintering, or powder, wire, or sheet metal melting.

In certain embodiments, the tantalum, niobium, zirconium, zinc, tungsten, lithium, potassium, strontium, sodium, calcium, chromium, molybdenum, tin, manganese, iron, or cobalt is present in an un-melted form.

In certain embodiments, the tantalum, niobium, zirconium, zinc, tungsten, lithium, potassium, strontium, sodium, calcium, chromium, molybdenum, tin, manganese, iron, or cobalt is fully melted and alloyed with the titanium and copper.

In certain embodiments, the tantalum, niobium, zirconium, zinc, tungsten, lithium, potassium, strontium, sodium, calcium, chromium, molybdenum, tin, manganese, iron, or cobalt is present homogeneously throughout the bulk of the implant part alloy.

In certain embodiments, the tantalum, niobium, zirconium, zinc, tungsten, lithium, potassium, strontium, sodium, calcium, chromium, molybdenum, tin, manganese, iron, or cobalt is distributed locally in a specific area(s) of the implant part alloy.

Illustrative embodiments are described below in the following numbered paragraphs:
1. A titanium-based biocompatible metal alloy, comprising:
an amount of titanium, aluminum and vanadium;
wherein the amount of aluminum in the biocompatible metal alloy is in a range from about 1% to about 4.5% of the titanium amount, and wherein the vanadium amount varies from about 0.5% to about 3% of the titanium amount.
2. The titanium-based biocompatible metal alloy of paragraph 1, wherein the biocompatible metal alloy is configured as a medical implant shaped for a subject body part.
3. The titanium-based biocompatible metal alloy of paragraph 1, wherein the biocompatible metal alloy is configured as an industrial component.
4. The titanium-based biocompatible metal alloy of paragraph 1, wherein the biocompatible metal alloy is processed via laser-based metal deposition in a controlled oxygen environment and coated on a desired metallic implant shaped for a subject body part.
5. The titanium-based biocompatible metal alloy of paragraph 1, wherein the composition is processed via electron beam-based or plasma-based or electrical arc melting-based metal deposition in a controlled oxygen environment and coated on a desired metallic implant shaped for a subject body part.
6. The titanium-based biocompatible metal alloy of paragraph 1, further comprising at least one metallic element selected from: tantalum, niobium, zirconium, zinc, tungsten, lithium, potassium, strontium, sodium, calcium, chromium, molybdenum, tin, manganese, iron, and cobalt.
7. The titanium-based biocompatible metal alloy of paragraph 6, wherein the amount of at least one metallic element of the biocompatible alloy can be varied in a range from about 0.00 to about 50%.
8. The titanium-based biocompatible metal alloy of paragraph 1, wherein the biocompatible metal alloy is further alloyed from at least one oxide selected from: magnesium oxide, silicon oxide, calcium oxide, strontium oxide, zinc oxide, tungsten oxide, lithium oxide, potassium oxide, sodium oxide, chromium oxide, molybdenum oxide, tin oxide, manganese oxide, iron oxide, and cobalt oxide.
9. The titanium-based biocompatible metal alloy of paragraph 8, wherein the amount of at least one oxides of the biocompatible alloy can be varied in a range from about 0.00 to about 50%.
10. The titanium-based biocompatible metal alloy of paragraph 1, wherein the biocompatible metal alloy includes copper and zinc.
11. The titanium-based biocompatible metal alloy of paragraph 10, wherein the amount of copper is in a range from about 0.01 to about 30%.
12. The titanium-based biocompatible metal alloy of paragraph 10, wherein the amount of zinc is in a range from about 0.01 to about 30%.

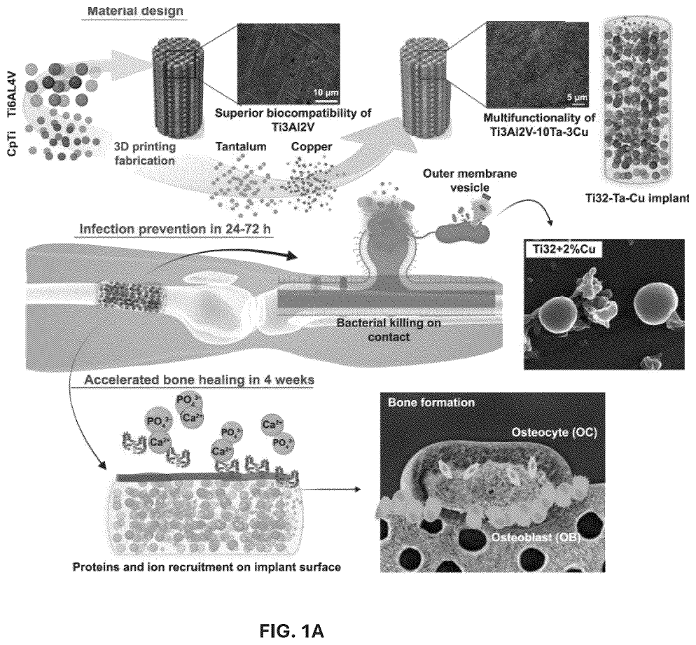

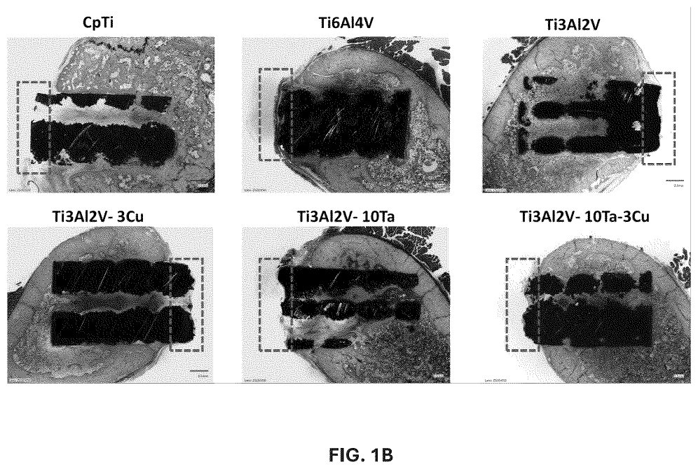

Turning specifically to the drawings, FIG. 1 shows a schematic of synergistic antibacterial efficacy and enhanced biocompatibility of Ti3Al2V—Cu—Ta alloys toward early-stage osseointegration. These implants, as a beneficial example methodology, are fabricated via multi-material 3D Printing or additive manufacturing to impart continued bacterial resistance while enabling early bone formation through enhanced biocompatibility. In this light, fabricated Ti3Al2V, as disclosed herein, is achieved by physically pre-mixing CpTi and Ti6Al4V powder in equal weight proportion using metal additive manufacturing to evaluate the mechanical and biological response of processed dense and porous parts. Following this, multi-material additive manufacturing is employed to alloy Ti3Al2V with, for example, about 2 and 3% Cu for antibacterial efficacy evaluation.

### Materials and Methods

### Design and Fabrication: Selective Laser Melting (SLM)

CpTi and Ti6Al4V compositions were printed on a laser powder bed fusion (LPBF) system. A third composition was prepared and printed by mixing CpTi and Ti6Al4V powders in a 1:1 weight ratio, i.e., Ti3Al2V, since the amounts of Al and V were reduced by 50% each to 3 and 2 wt. %, respectively. Individual dense and porous (20 and 40% volume fraction porosity) cylindrical structures were designed for compression and shear strength measurements at the porous-dense interface. Alloy compositions Ti3Al2V-2Cu, Ti3Al2V-3Cu, Ti3Al2V-10Ta, and Ti3Al2V-3Cu-10Ta with 40% volume fraction porosity were built using LPBF for in vitro bacterial resistance studies and in vivo biological response studies. All structures were fabricated on a selective laser melting (SLM)-based PBF system (3D Systems ProX® DMP 200, Rock Hill, SC) with a 300 W fiber laser and wavelength 2=1070 nm. The system has a powder supply chamber and a melting stage. Spherical metal powders were used. CpTi powders were procured from GKN Hoeganaes (Cinnaminson, NJ, USA) and Ti6Al4V from AP&C (GE Additive, Cincinnati, Ohio, USA). Metal powders were sieved to obtain a particle size of <63 μm. These metal powders were added to the supply side. A CpTi build plate of ˜2.5 cm thickness was used and placed on the melting stage. The build chamber was enclosed and purged with argon gas with O₂<500 ppm.

Additive manufacturing of Ti6Al4V and CpTi is widely used. The optimized laser power and scan speed parameters are 180 W and 1600 mm/s, respectively, for successful fabrication using a powder bed fusion (PBF) process, which was used for compositions CpTi, Ti6Al4V, Ti3Al2V, and Ti3Al2V-3Cu. With the addition of alloying elements, the part quality and print resolution varied depending on intrinsic material properties like heat diffusivity and laser absorption. Ta and Cu demonstrate contrasting laser-material interactions. Ta shows excellent laser absorption but has a very high melting point of 3017° C., indicating higher energy input for successful PBF. However, Cu is a poor laser absorber, reflecting 98% of the laser wavelengths in 1000-1100 nm range. Cu also demonstrates more than two times the viscosity and 100 times heat diffusivity in the molten state compared to Ti, dictating the higher energy required for the laser-PBF operation. An in-depth study on the powder bed fusion printing optimization for these alloys resulted in a laser power of 196 W and a scanning speed of 1440 mm/s to be the optimal printing parameters used to fabricate these compositions.

A layer thickness of 30 μm was used for each layer. The hexagonal laser scan strategy was used for dense and strip laser scan strategy for printing porous structures. All compositions were printed with the same print parameters (denoted as ‘as-printed’ structures hereafter). Post printing, structures were cut from the build plate and ground on 120 grit SiC papers to make the opposite surfaces parallel, followed by multiple sonication treatments in deionized (DI) water and ethanol, then compressed air spraying to remove all loose powders inside the pores. Bulk volume porosities were calculated by taking the measured volume of the structures to the theoretical volume of their respective dense composition. More information on design and porosity evaluation is presented in the supplemental file. Rectangular cross-section samples were manufactured for wear studies. Some of those samples were fabricated on a directed energy deposition (DED)-based AM system (FormALLOY, CA) and described briefly in the supplemental file.

### Phase Analysis, Microhardness, and Microstructure

Phase detection was done using x-ray diffraction. Vickers microhardness tests were performed on the surface in the x-y plane of the build direction. Microstructure was observed by cutting longitudinally along the build direction. The sections were mounted in a phenolic resin followed by grinding on 80-1200 grit size SiC grinding papers and polishing using 0.05-1 μm of suspended alumina in deionized water (DI). The Vickers microhardness test was conducted on a Phase II Plus Micro Vickers Hardness tester (Upper Saddle River, NJ, USA) using a load of 200 gms and a dwell time of 15 s. A total of n=15 microhardness measurements were conducted for each composition. Surfaces were etched in Kroll's reagent for 45 s, and microstructures were observed under a Scanning Electron Microscope (SEM, Apreo, Thermo Scientific, MA, USA).

### Mechanical Strength Evaluation: Compression, Shear, and Fatigue

As per ASTM E9-19, dense and porous cylindrical structures for all compositions with 7 mm diameter and ˜15 mm height were subjected to compression testing on Instron servo-hydraulic machine (600DXS, Grove City, Pennsylvania). At least 3 replicates were tested for each composition and porosity, respectively. A crosshead displacement rate of 1.3 mm/min was used across the compositions. The corresponding load-displacement data was recorded. Elastic modulus was calculated from the linear slope region from the stress-strain curve. Compressive yield strength was evaluated using the 0.2% strain offset method. Shear strength was evaluated at the porous-dense interface using a single-shear test device developed in our lab per the procedure described in reference. Structures with 3.1 mm diameter and ˜12 mm height were used. The nature of the shear test carried out is tensile rather than torsional. The porous-dense interface was placed at the interface of the shear plates and pulled in tension in opposite directions with a crosshead displacement rate of 0.3 mm/min on an Instron servo-hydraulic testing machine (600DXS, Grove City, Pennsylvania). An external 1360 kg load cell and an extensometer were used to precisely measure the load and displacement of the shear plates, respectively. Structures were sheared till fracture, and the corresponding load and displacement data were recorded. At least three replicates were tested for each composition and porosity, respectively. The shear modulus was evaluated as the slope of the linear region in the shear stress-strain curve. Maximum shear strength was evaluated as the highest shear strength endured by the structure before failure. Fractured surfaces at the porous-dense interface were observed under a Scanning Electron Microscope (SEM, Apreo, Thermo Scientific, MA, USA). No etching was done on fracture surfaces for microstructural imaging. The fatigue tests were performed on an ADMET eXpert 9300-Rotating Beam Fatigue system (Norwood, MA). The Rotating Beam tester applies a force via a bending moment to induce surface stress on a sample. Each surface experiences tensile and compressive stresses as the sample rotates until failure. The digital controller displays the number of cycles for the sample to fail.

### Wear and Open Circuit Potential (OCP) Tests

In vitro tribological testing was carried out using a ball-on-flat set up on ground-polished DED printed CpTi, Ti6Al4V, Ti3Al2V, Ti3Al2V-3Cu, Ti3Al2V-10Ta and Ti3Al2V-10Ta-3Cu samples following ASTM G133-05 [17]. The tests were done using a Biotribometer (Ducom, India) in Dulbecco's Modified Eagle's medium (DMEM) (Sigma-Aldrich) with a 5 N applied load, 3 mm diameter zirconia (ZrO2) wear ball, translation speed of 72 m/h, and a 10 mm amplitude for a total sliding distance of 1000 m. Compound wear (CW) and coefficient of friction (COF) were recorded; compound wear for the tested samples was attained using the built-in linear variable differential transducer (LVDT) to measure the z-axis displacement of the tribological loading arm throughout testing. The measurement considers both wear on the counter wear ball and the tested sample, hence the compound wear. A 2-electrode corrosion-cell configuration acquired the open circuit potential (OCP) with a modular line Metrohm Autolab potentiostat/galvanostat (Riverview, FL, USA). The fabricated structures were used as the working electrode (WE), and the reference electrode (RE) was a saturated Ag/AgCl/KCl. The structures were immersed in DMEM and allowed to stabilize for a minimum of 2-3 h before starting the tribological wear test. The tracks on the samples and scars on the ZrO2 wear balls were imaged under a Scanning Electron Microscope (Quanta 200F, Thermo Fisher, Waltham, MA) and optical microscope, respectively.

### In Vivo Study

Both dense and porous (40% volume fraction porosity) CpTi, Ti6Al4V, Ti3Al2V, Ti3Al2V-3Cu, and Ti3Al2V-10Ta-3Cu compositions were used for the in vivo biological response study. In vivo studies were designed with a 2-phase parallel evaluation. First, CpTi and Ti6Al4V were considered controls to evaluate the biological response of Ti3Al2V compositions. This phase was designed to assess whether Ti3Al2V had similar or better osseointegration than the already established CpTi without compromising the mechanical tissue-material fixation that Ti6Al4V offers. The second phase was a post-Ti3Al2V assessment to evaluate toxicity, enhancement in biological response, and osseointegration of Ti3Al2V-3Cu and Ti3Al2V-10Ta-3Cu over Ti3Al2V.

### Surgery and Implantation Procedure

Male Sprague-Dawley rats with average weights between 300-350 gms were used for the in vivo study. The rats were acclimatized in separate cages in a temperature and humidity-controlled room for at least a week before the surgeries. The animals were administered buprenorphine (0.03 mg/kg) 30 minutes before anesthesia as a pain-reducing medication. The animals were anesthetized with a prescribed dose of IsoFlo® (isoflurane, USP, Abbott Laboratories, North Chicago, IL, USA) coupled with oxygen (Oxygen USP, A-L Compressed Gases Inc., Spokane, WA, USA) and periodically monitored by respiration rate during the surgery. Once anesthetized, the animals were shaved around the implantation area and cleaned thrice with alternating chlorhexidine and isopropyl alcohol scrubs. As a numbing agent, 0.3 ml of Lidocaine HCL (without epinephrine), 0.5% was administered subcutaneously on each leg near the incision area.

An incision was made on the lateral side above the distal femoral condyle, and a unicortical defect of 2.5 mm diameter was made on the lateral epicondyle using the gradually increasing diameter of drill bits. The defect site was rinsed with saline to prevent thermal necrosis and remove residual bone fragments, and the implant was placed in the defect. The fascia over the incision, followed by the skin, was then sutured using undyed braided coated MONOCRYL-polyglactin 910 (Ethicon Inc., Somerville, NJ, USA) outer skin was stapled using sterile surgical staples. An anti-inflammatory analgesic, meloxicam (0.2 mg/kg), and lactated ringer's solution-(LRS, 3 ml) for rehydration were administered post-surgery subcutaneously for the first 2-3 days, and the animals were monitored until they regained consciousness. Postoperative care was carried out for 3 days, with buprenorphine administration every 12 h and meloxicam every 24 h. After 6 weeks, the rats were euthanized by carbon dioxide overdose, followed by cervical dislocation as a secondary measure. The harvested metal-bone explants were fixed in 10% neutral buffered formalin for at least 72 h for tissue infiltration. The Institutional Animal Care and Use Committee (IACUC) of Washington State University (Pullman, WA) approved protocol was followed to perform the experimental and surgical procedure.

### Histological Analysis

After fixing the explants in 10% neutral buffered formalin for 72 hours, serial dehydration was carried out in ethanol and embedded in polymethyl methacrylate (PMMA).

They were then sliced into thin sections along the longitudinal direction of the metal implantation and the surrounding bone using a Exakt™ saw, ground, and mounted on glass slides. Sanderson's Rapid Bone Staining (SRBS) and Hematoxylin and eosin (H&E) staining were carried out on separate glass slides for each composition for histological analysis. These stained bone sections were then observed under a Keyence digital microscope (Model VHX-7000, Itasca, IL), exploiting the microscope's multi-lighting and 3D-depth composition features to observe osteoid and trabecular bone formation at the bone-material interface.

### Histomorphometry Analysis

The stained sections were imaged under a Keyence digital microscope (Model VHX-7000, Itasca, IL), and histomorphometry analysis was carried out using the microscope's multi-lighting and 3D-depth composition features, which allowed for accurate spherical rendering and detailed imaging of the histological features to the slide thickness. SRBS-stained slides were observed for osteoid and trabecular bone. H&E-stained sections were analyzed for any visible markers of the inflammatory response. Quantitative osseointegration at the bone-implant interface was inspected based on modified scoring criteria per, as shown in Table 1.

TABLE 1
Modified scoring for quantitative osseointegration at the bone-implant
interface following criteria per ISO 10993: 6 (2016) Annex E.2

| | Score | | | | |
|---|---|---|---|---|---|
| Parameter | 0 | 1 | 2 | 3 | 4 |
| Trabecular | Absent | Minimal, | Mild, | Moderate, | Marked, |
| apposition | | 1-25% | 26-50% | 51-75% | 76-100% |
| Fibrocar- | Absent | Minimal, | Mild, | Moderate, | Marked, |
| tilage | | 1-25% | 26-50% | 51-75% | 76-100% |
| presence | | | | | |
| Osteoid at | No | osteoid | Mostly | A mix of | Mostly |
| interface | bone or | | woven | woven and | lamellar |
| | osteogenic | | bone | lamellar | bone |
| | islands | | | bone | |
| fibrosis | absent | narrow | moderately | Thick | extensive |
| | | band | thick band | band | band |
| tissue | absent | minimal | mild | moderate | marked |
| ingrowth | | | | | |
| into the | | | | | |
| device | | | | | |

The modifications in the scoring criteria were decided based on parameters relevant to the scope of this research. The final scoring parameters were trabecular apposition, osteoid at the interface, fibrocartilage presence, inflammation (from H&E-stained sections), fibrosis, and tissue ingrowth into the implant. Out of these six parameters, the first three were quantified as the average area fraction of each parameter calculated using Trainable Weka Segmentation in ImageJ over 7 different sections under a high-power field (×1000) around the BIC region. Individual sections were further divided into 4 segments for the Random Forest Algorithm. The other four parameters were qualitatively evaluated from low-power field images of the tissue sections (See FIG. 1B). In particular, FIG. 1B shows histology micrographs of tissue cross-sections at the bone-implant interface stained with Sandersons Rapid Bone Stain (SRBS). Low power field images shows favorable fibrocartilage lining at the lateral edge of the implants (box) in all compositions except Ti6Al4V which shows a clear fibrous tissue.

### In Vitro Bacterial Study

Bacterial culture was carried out for CpTi, Ti6Al4V, Ti3Al2V, Ti3Al2V-2Cu, and Ti3Al2V-3Cu using two relevant bacterial strains: Pseudomonas aeruginosa gram-negative bacteria for 36 h and Staphylococcus aureus gram-positive bacteria for 24 and 48 h Both strains of bacteria are relatively common in post-surgical orthopedic infections. Freeze-dried P. aeruginosa (Carolina Biological, NC) and S. aureus were rehydrated using rehydration media. Subsequently, dilutions in nutrient broth were made for 0.5 McFarland standard optical density measurement, the correct dilution of 106 CFU/ml. Disc samples were sterilized and studied in triplicate for agar plate colony count and duplicates for SEM characterization. Samples were placed in separate wells in 24 well-plate, and 106 CFU/ml of both bacterial colonies were seeded on the autoclaved polished surface augmented with 2 ml of nutrient broth per well. After respective time points, bacterial cells were scraped from the surface of the 3 out of 5 samples using cell scrapers, mixed in 2 ml 0.1M phosphate buffer saline (PBS), and serially diluted to approximately contain colonies between 30 and 300. 1 μl of the respective solutions were streaked on a cetrimide agar plate (Pseudosel agar, Fisher Scientific, NH) for P. aeruginosa and tryptic soy agar plates for S. aureus. Bacterial colonies on agar plates were counted after 24 hrs of incubation, and the antibacterial efficacy was evaluated as a function of bacterial colonies on individual material compositions as:

$$N = C \times d \times 10 \; 00 / l$$

$$R = ( {N}_{contro} l - {N}_{material} ) / {N}_{control} \times 100 \% ,$$

Where N is the calculated number of bacterial colonies observed, C is the average colony count on a plate, d=dilution factor, and 1=volume of bacterial suspension on the sample. To study the bacterial cell morphology, SEM samples were preserved in 1% glutaraldehyde and 1% paraformaldehyde in 0.1M phosphate buffer overnight, followed by dehydration, gold coating, and imaging was carried out on a Scanning Electron Microscope (SEM, Apreo, Thermo Scientific, MA, USA).

### Results

The impact of the chemistry and structural design of 3D-printed Ti3Al2V alloys were evaluated for their mechanical and biological performance compared to CpTi and Ti6Al4V. Bulk porosity for as-printed dense and porous structures was evaluated separately using the same structures used for compression testing vis-à-vis Ti6Al4V and Ti3Al2V. For Ti6Al4V, the % bulk porosities were measured to be 3.7, 14.8, and 35.8%, denoted as Ti6Al4V-D, Ti6Al4V-P20, and Ti6Al4V-P40, respectively. Porosities for Ti3Al2V were 3.8, 19.2, and 42.9% and were denoted as Ti3Al2V-D, Ti3Al2V-P20, and Ti3Al2V-P40, respectively. The 3.6 and 3.8% porosities for Ti6Al4V-D and Ti3Al2V-D are residual. Although optimized processing parameters were used for all compositions, minor variations in porosities were observed due to a change in chemistry and related variations in laser absorptivity during processing.

### Phase Analysis, Hardness, and Microstructure

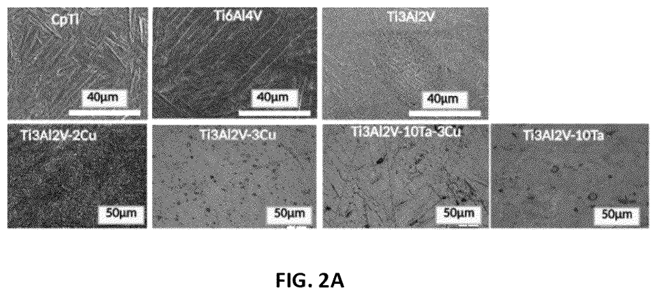

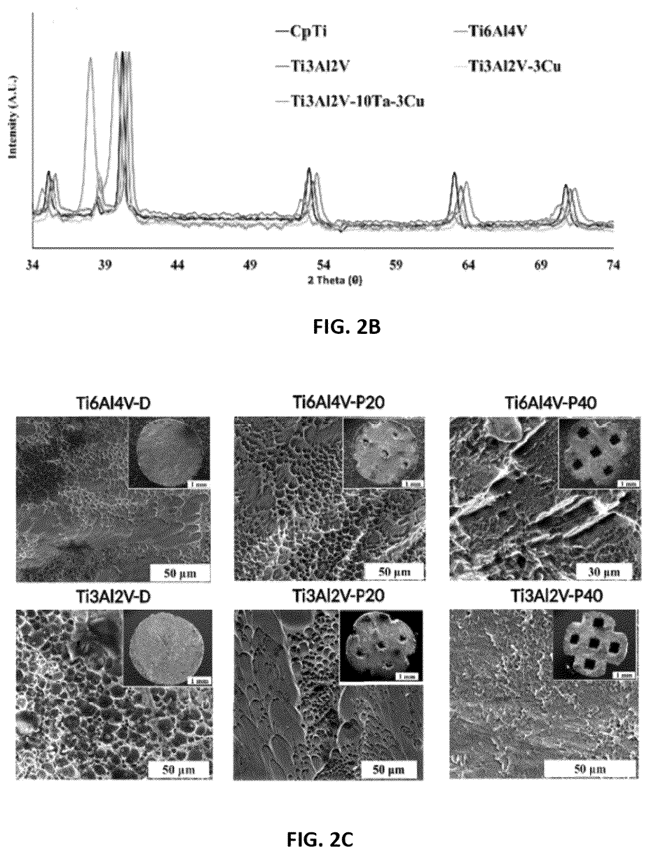

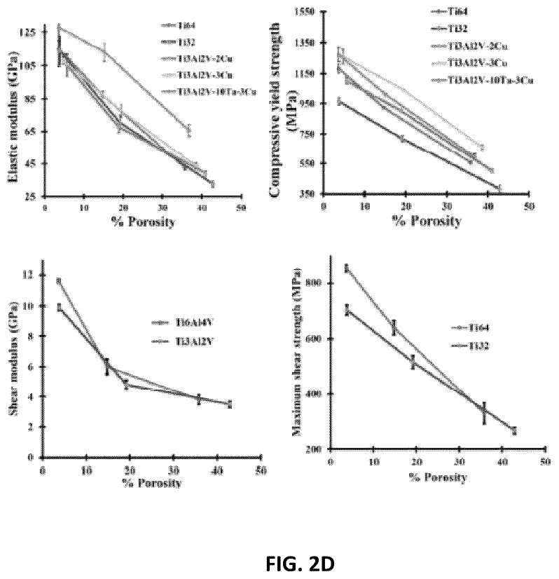

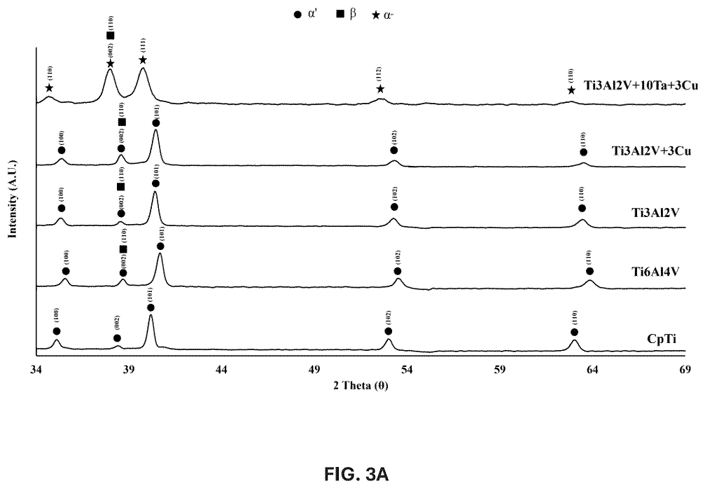

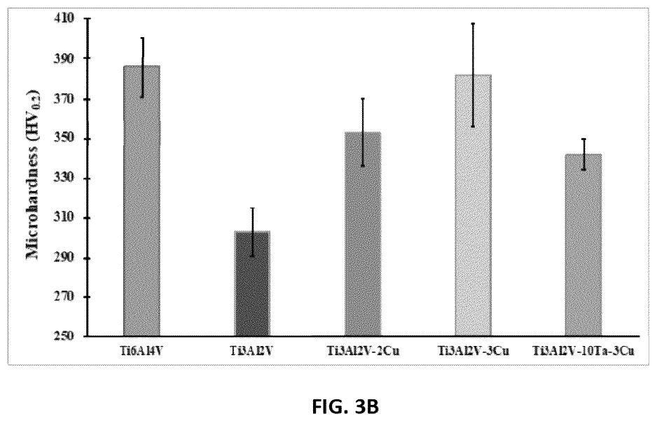

FIGS. 2A-2D SEM show micrographs and analysis of the etched surfaces for all the compositions. FIG. 2A specifically shows highly anisotropic acicular α′ martensitic needles for Ti3Al2V compared to CpTi and Ti6Al4V, which show typical α and β microstructures, respectively. X-ray diffraction was used to identify phases in these compositions. Microstructural analysis reveals that the acicular needle-like characteristic phase is present even for Ti3Al2V-2Cu composition; however, adding more Cu content and 10% Ta resulted in the refinement of grains and a change in microstructure from needle-like to more lamellar, which is consistent with a solid-solution microstructure of Ti—Ta alloys. The most prominent X-ray diffraction peaks observed for all compositions are for α-Ti, see FIG. 2B (also see FIG. 3A). No prominent β-Ti peaks were observed, similar to previously reported results. We observe a shift in the peaks for Ti6Al4V and Ti3Al2V towards the right compared to CpTi peaks due to alloying elements Al and V, making them an α+β-Ti alloy as opposed to α-Ti in CpTi. This shift is higher for Ti6Al4V compared to Ti3Al2V due to the presence of a higher amount of β-Ti. Comparing the peak intensities, for all the peaks except α-Ti (002), the highest intensity was observed for CpTi, followed by Ti3Al2V, and the lowest for Ti6Al4V. Although the difference in the peak intensities is insignificant, the peaks for CpTi show the highest peak intensity values due to the higher amount of α-Ti presence. Comparing Ti6Al4V and Ti3Al2V, due to lower contents of alloying elements of Al and V in the latter, the amount of α-Ti is higher in Ti3Al2V than that of Ti6Al4V. Only for the α-Ti (002) the trend in the peak intensities is reversed. The highest peak is observed for Ti6Al4V, followed by Ti3Al2V, and the lowest for CpTi. We hypothesize that this is a combination peak intensity of the β-Ti peak (011) and the α-Ti peak for (002), as also observed by other research groups. For Ti3Al2V-3Cu, all the α′ peaks, except α′(002) and α′(101), observe a rightward shift and lower peak intensities for α′—Ti in comparison to those Ti3Al2V indicating increased β-Ti phase since Cu is a β-Ti stabilizer. For α′(002) peak, we observe a higher intensity peak for Ti3Al2V-3Cu than that for Ti3Al2V. As mentioned, this peak coincides with the β-Ti (110) peak, indicative of a higher β-Ti phase in Ti3Al2V-3Cu. No Cu peaks were observed. For Ti3Al2V-10Ta-3Cu, we observed a leftward shift for all the peaks due to Ta addition causing lattice strains since Ta has a higher lattice parameter than Ti. The phases formed in this case are α″—Ti due to rapidly quenched supersaturated bcc-Ti—Ta solid solution. The α″(002) coinciding with the peak has a very high intensity in the β(110) peak, indicating a high amount of β-Ti formation since Ta is also a β-Ti stabilizer. Vickers microhardness values for CpTi, Ti6Al4V, and Ti3Al2V were 264±11, 386±15, and 303±12 HV0.2, respectively. Ti3Al2V followed the highest hardness value of Ti6Al4V due to the latter's higher content of alloying elements. Hardness values for Ti3Al2V-2Cu and −3Cu were 353±17 and 382±26 HV0.2, respectively. The hardness enhancement is due to the formation of intermetallic Ti2Cu and solid solution strengthening by Cu solute atoms; the higher the amount of Cu, the higher the hardness value observed (2 vs.3% Cu). The hardness value for Ti3Al2V-10Ta-3Cu was 342±8 HV0.2, indicating Ta did not increase hardness (also see FIG. 3B).

### Mechanical Strength Characterization: Compression, Shear, and Fatigue

Metallic implants at load-bearing sites are constantly under multi-axial loading in vivo. It becomes essential to understand the mechanical behavior of such implants under different loading conditions to minimize the possibility of implant failures. Dense and porous samples for all compositions were subjected to compression loading, while dense-porous interface samples for Ti6Al4V and Ti3Al2V were subjected to shear loading, and only dense samples were subjected to fatigue loading. AM-processed Ti6Al4V was used as a positive control.

### Shear Behavior

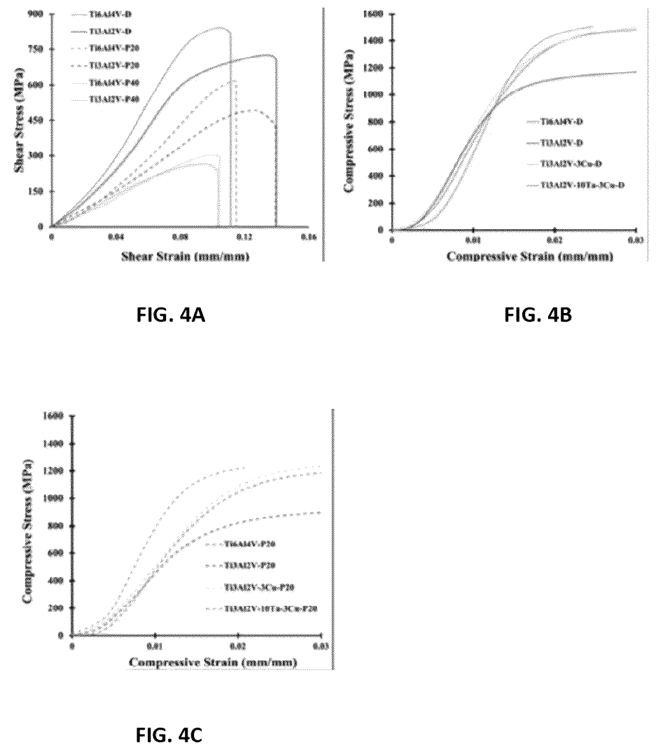

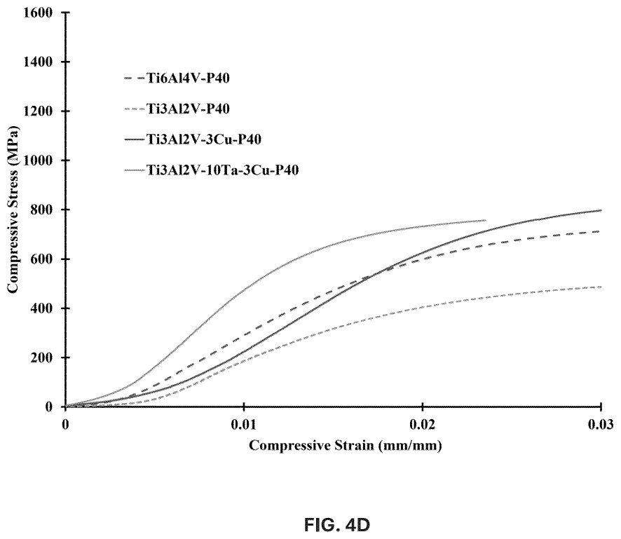

The weakest point lies in the porous-dense interface material design for porous coatings on the bulk implant. For Ti3Al2V to be a more versatile material, it is essential to evaluate the shear characteristics at this porous-dense interface to prevent coating failures in such coating applications [28]. SEM micrographs at the porous-dense failure interface, FIG. 2C, reveal ductile dimples for both compositions, indicating a desirable ductile behavior. It can be corroborated from the raw shear stress-strain plots, (see FIG. 4A), Ti3Al2V-D shows higher ductility than Ti6Al4V-D before failure, and the same for Ti3Al2V-P20 and Ti6Al4V-P20. Even with increased porosity at the porous-dense interface, the ductility was comparable for Ti3Al2V-P40 and Ti6Al4V-P40. Shear stress-strain plots from tests are presented in (see (see FIG. 4A). Shear modulus and maximum shear strength are plotted against their porosities at the porous-dense interface in FIG. 2D. It should be noted that the nature of the shear test performed is tensile rather than torsional. Reduced shear modulus and strength were observed at the porous-dense interface from Ti6Al4V to Ti3Al2V. Shear modulus for dense structures Ti6Al4V-D and Ti3Al2V-D were 11.6 and 9.9 GPa, i.e., reduced Al and V content from Ti6Al4V-D to Ti3Al2V-D led to a 15% reduction in shear modulus. Thus, it was observed that the change in shear modulus was a function of the compositions. Maximum shear strength endured by Ti6Al4V-D and Ti3Al2V-D were 853 and 705 MPa; a 17% reduction in maximum shear strength was observed with a reduction in Al and V. For the P20 group, shear modulus for Ti6Al4V-P20 (6 GPa, 14.8% porosity) was higher than that for Ti3Al2V-P20 (4.8 GPa, 19.2% porosity), and the shear strengths were 640 and 516 MPa, respectively; 20% shear modulus reduction and 19% shear strength reduction from Ti6Al4V-P20 to Ti3Al2V-P20 was observed, given that Ti3Al2V-P20 had a 4.4% higher porosity. For the P40 group, the shear modulus difference between Ti6Al4V-P40 (3.8 GPa, 35.8% porosity) and Ti3Al2V-P40 (3.5 GPa, 42.9% porosity) decreased; only an 8% reduction in shear modulus was observed from Ti6Al4V-P40 to Ti3Al2V-P40, given that Ti3Al2V-P40 had 7.1% higher porosity. Comparing shear strength values for Ti6Al4V-P40 (330 MPa, 35.8% porosity) and Ti3Al2V-P40 (267 MPa, 42.9% porosity), a reduction of 19% in shear strength was observed from Ti6Al4V to Ti3Al2V, given that Ti3Al2V-P40 had 7.1% higher porosity than Ti6Al4V-P40. It can be seen from FIG. 2D that with an increase in porosity at the maximum strength, the reduction was steeper for Ti6Al4V compared to Ti3Al2V.

### Compressive Behavior

It is desired for an implant material to demonstrate an elastic modulus closer to that of the natural bone (˜5-30 GPa), with high yield strength. This reduction in elastic modulus is achieved by introducing controlled porosity in metallic implants. Elastic modulus and compressive yield strength values for dense and porous Ti6Al4V and Ti3Al2V against their evaluated porosities are plotted in FIG. 2D. CpTi and Ti6Al4V display similar elastic modulus values, ˜110-114 GPa. Since Ti3Al2V is a mixture of the two compositions, it is not expected that Ti3Al2V's elastic modulus to vary compared to Ti6Al4V. The variation in elastic modulus between Ti6Al4V and Ti3Al2V can be attributed primarily to the porosity difference. The elastic modulus for Ti6Al4V-D and Ti3Al2V-D were similar, 115.3 and 114.1 GPa, respectively. For the P20 structures, Ti6Al4V-P20 (porosity 14.8%) showed an elastic modulus of 87.2 GPa, and that for Ti3Al2V-P20 (19.2%) was 70.3 GPa. Similarly, for P40 structures, the modulus for Ti6Al4V-P40 (porosity 35.8%) was 42.3 GPa, and that for Ti3Al2V-P40 (porosity 42.3%) was 32.5 GPa. Dense Ti3Al2V-3Cu showed a modulus of 108.2±2.1 GPa, and when Ta is added to Ti3Al2V-3Cu to form Ti3Al2V-10Ta-3Cu, the elastic modulus was observed to be 144.3±10.2 GPa. Cu and Ta are b-Ti phase stabilizers, but the high cooling rates of AM prevented the formation of the b-Ti phase, resulting in no effect of lower modulus value by Cu addition (pure Cu modulus ˜130 GPa) and modulus enhancement due to Ta addition since pure Ta has a modulus of ˜185 GPa. As stated before, minor porosity variations happened due to compositional variations, even with optimized processing parameters.

As opposed to the high compressive yield strength of Ti6Al4V˜1100 MPa, CpTi shows a yield strength as low as 432 MPa. This enhanced strength in Ti6Al4V is due to the addition of Al and V solute atoms inducing the formation of a high-strength-low modulus β-Ti phase in Ti6Al4V. The variation in compressive yield strength can be attributed to the composition and porosity. An 18% reduction in strength from Ti6Al4V-D (1181 MPa) to Ti3Al2V-D (965 MPa) was seen due to reduced Al and V amounts. From Ti6Al4V-P20 (922 MPa) to Ti3Al2V-P20 (719 MPa), a 22% reduction in compressive yield strength, with 4.4% higher measured porosity for Ti3Al2V-P20 than Ti6Al4V-P20 is observed. Similarly, Ti6Al4V-P40 (557 MPa) to Ti3Al2VP40 (382 MPa), with 7.1% higher porosity for Ti3Al2V-P40, reduced compressive yield strength by 31%. At the same time, the strength observed for Ti3Al2V-P40 (382 MPa, 42.9% porosity) is comparable to that of dense CpTi (˜350 MPa). For Ti3Al2V-10Ta-3Cu, the compressive yield strength increased to 1255±51 MPa. This enhancement in strength is due to the formation of Ti2Cu intermetallic formation in Ti3Al2V-10Ta-3Cu alloy and the solid solution strengthening effect due to Cu. Raw stress vs. strain plots for each porosity and composition are reported in (see FIG. 4B-D).

### Fatigue Behavior

AM-processed dense fatigue samples of Ti6Al4V, Ti3Al2V, and Ti3Al2V-10Ta-3Cu at a 90° build angle were tested using an ADMET eXpert 9300-Rotating Beam Fatigue testing system. The 90° build angle is tested under fatigue loading as that is the weakest build direction due to being parallel to the loading direction. The fatigue specimens were turned to the final shape to minimize AM-generated surface roughness at the gauge length. Samples were then heat treated at 400° C. for 1 h and cooled slowly in the furnace to reduce residual stresses. After removal from the furnace, the sample is polished using emery cloth ranging from 400-600 grits until no defects are visible on the surface. Samples were tested to determine at what stress amplitude samples can survive at least 10 million cycles without failure. Ti6Al4V and Ti3Al2V samples survived 10 million cycles at 21% of their respective compressive yield strength. Anisotropy is an important factor in additively manufactured structures. Ti3Al2V structures at 0° build angle demonstrated almost two times higher fatigue endurance limit than Ti3Al2V at 90°. With a further increase in stress amplitude, samples failed at lower cycles. For the Ti3Al2V-10Ta-3Cu samples, no failure up to 10 million cycles was accomplished at 19% of the compressive yield strength. Our initial fatigue test results indicate that lowering Al and V in Ti and adding Ta and Cu do not degrade the excellent fatigue response of these alloys.

### Tribological Study

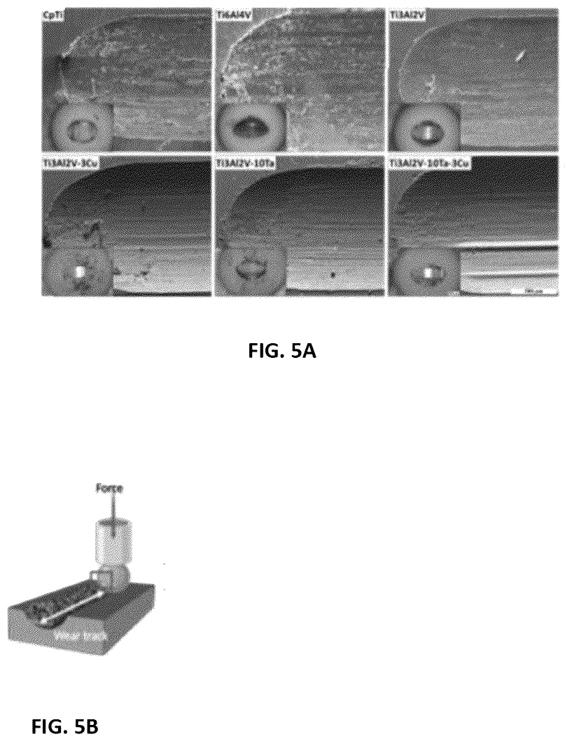

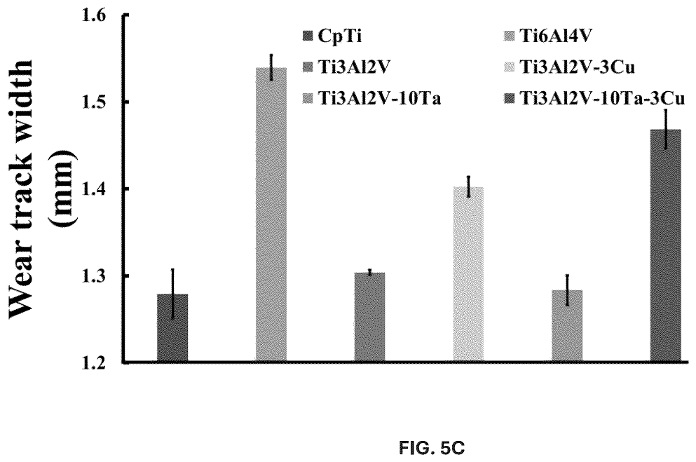

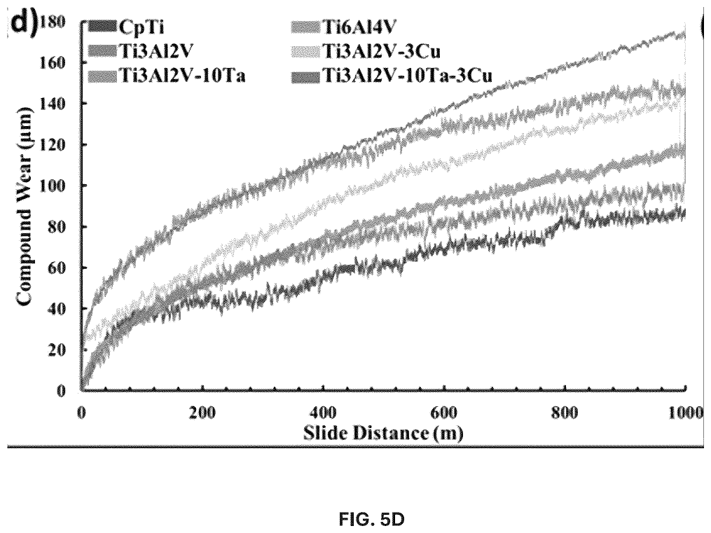

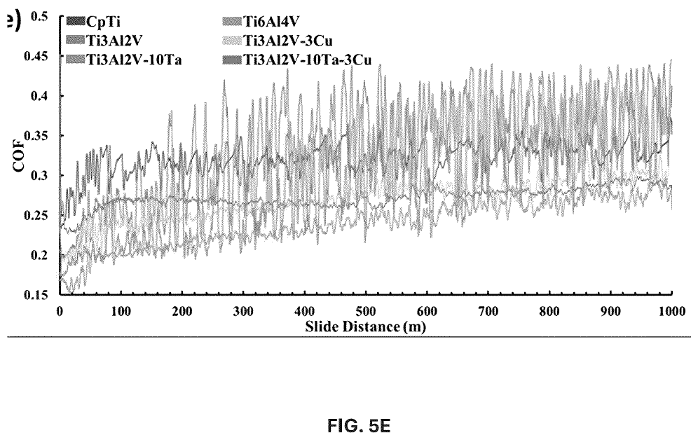

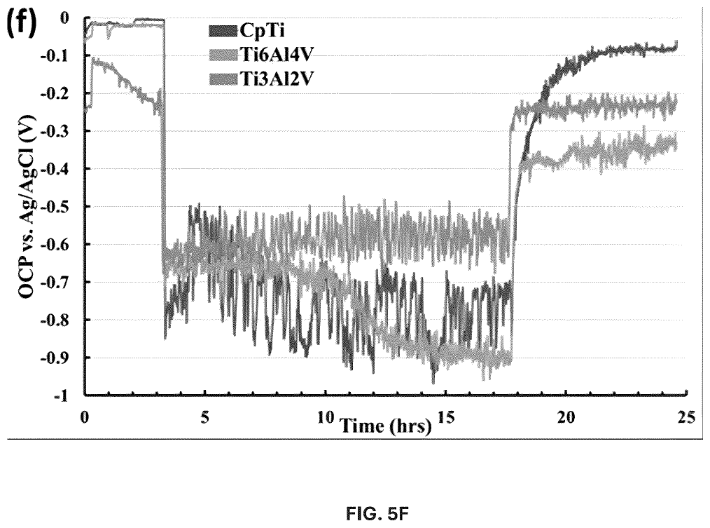

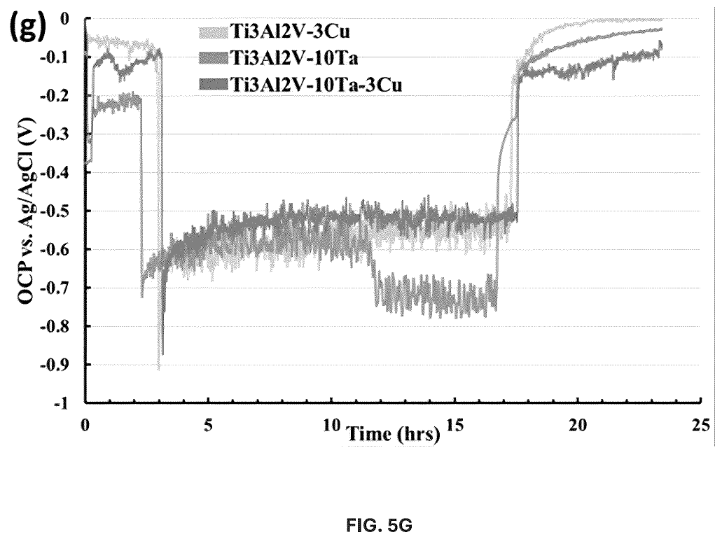

Tribological testing and characterization are essential to any material for physiological load-bearing sites. Proper interpretation of the physical wear phenomena must be evaluated from the compound wear (CW) curve, coefficient of friction (COF), and worn surface imaging of the tested sample and the counter wear material; this is needed to determine the wear-induced degradation and material deformation thoroughly. Additionally, an investigation into the electrochemical passive nature of the material during tribological testing should be done. Such testing requires a data logging unit, known as a potentiostat, a working electrode (WE), a reference electrode (RE), and an electrically conductive media or an electrolyte. The results of such tribo-corrosive testing allow for quantification and further understanding of the material's chemical behavior or evolution during tribological testing; chemical changes usually occur within the passivation, de-passivation, and re-passivation domains. In the presence of physiological or simulated body fluid, such as electrolytes, the testing can be in vitro. The bio-tribo-corrosive results for these alloys are displayed in (see FIGS. 5A-G).

### Wear Behavior

Electron micrographs of the wear track surface and optical images of the counter wear ball were attained and are displayed in FIG. 5A. A design rendering of the physical interaction between the wear ball and sample is displayed in FIG. 5B. CpTi displayed the most gouging, plastic deformation, material removal and transfer, and subsequent deposition of material on the wear track itself; Ti6Al4V was comparable in appearance, while the Ti3Al2V wear track was smoother in appearance. Across all Ta and Cu compositions, a decrease in the material, adhesive transfer to the wear ball was observed, implying a suppression in the wear mode during tribological testing. When measuring the wear scar width on the ZrO2 wear balls, the values were in ascending order: 387 μm, 423 μm, 453 μm, 519 μm, 564 μm, and 657 μm for Ti3Al2V-10Ta-3Cu, Ti3Al2V-10Ta, Ti3Al2V-3Cu, Ti3Al2V, Ti6Al4V and finally CpTi, respectively. The measured values are proportional to the chord length associated with the arc length of the radial surface of interaction between the tested sample and the ZrO2 wear ball, i.e., the curvature of the wear track trough. A longer linear measurement is indicative of increased wear on the ZrO2. In the current study, the visible wear on the ZrO2 increases in the order of Ti3Al2V-10Ta-3Cu, Ti3Al2V-10Ta, Ti3Al2V-3Cu, Ti3Al2V, Ti6Al4V and finally CpTi, therefore Ti3Al2V-10Ta-3Cu displays the least and CpTi displays the most amount of wear on the ZrO2 counter wear ball. When measuring wear track width, Ti6Al4V displayed the greatest width at ˜1.55 mm, while CpTi, Ti3Al2V, and Ti3Al2V-10Ta had comparable wear track widths at ˜1.3 mm, as displayed in FIG. 5C. Final CW, as displayed in FIG. 5D, for the tested compositions was ˜175 μm, ˜147 μm, ˜143 μm, ˜117 μm, ˜98 μm and ˜88 μm for Ti3Al2V-10Ta-3Cu, Ti3Al2V-10Ta, Ti3Al2V-3Cu, Ti6Al4V, Ti3Al2V and CpTi, respectively. Ti3Al2V-10Ta and Ti3Al2V-10Ta-3Cu displayed the greatest running-in wear from 0-40 m. Amongst all 6 compositions, Ti3Al2V-3Cu, Ti3Al2V-10Ta-3Cu and Ti6Al4V exhibited the lowest COF, roughly 0.25 at 1000 m, as displayed in FIG. 5E. Although, Ti6Al4V remained with a constant positive slope while Ti3Al2V-10Ta-3Cu exhibited a steady-state near a moving average of zero slope in the COF.

### Open Circuit Potentials (OCPs)

The acquisition of the OCP before and during tribological testing results in the curves displayed in FIGS. 5F and G. The idle OCP (EIdle) is first attained when the sample is submersed into the media and is allowed to reach equilibrium potential. Once tribological testing commences, the OCP drops to anodic potentials concerning EIdle. The OCP attained at this instance is referred to as the wear OCP (EWear) and can dynamically change depending on a change in surface chemistry during tribological testing. Another essential quantification derived from a tribologically attained OCP curve is the ΔE potential (IEWear−Eldlel=ΔE)—the magnitude of the change in OCP from idle to wear conditions. OCP acquisition before tribological testing revealed that Ti6Al4V and CpTi initially demonstrated the most cathodic (positive) potential under idle conditions, with Ti3Al2V exhibiting the most anodic (negative), FIG. 5F. Upon tribological testing, an anodic shift across all compositions was observed, with Ti6Al4V exhibiting the most anodic shift and Ti3Al2V-10Ta-3Cu exhibiting the most cathodic, as displayed in FIG. 5G. As wear testing progressed, both CpTi and Ti6Al4V shifted to a more anodic potential relative to the start of the wear regime and when compared to Ti3Al2V, which shifted to a slightly more cathodic potential stabilizing to ˜−0.6 V. Ti3Al2V-3Cu and Ti3Al2V-10Ta-3Cu shifted to a more cathodic potential comparable to CpTi, Ti6Al4V and Ti3Al2V-10Ta. Upon unloading Ti3Al2V-3Cu, Ti3Al2V-10Ta, and Ti3Al2V-10Ta-3Cu re-passivated to more positive OCP values when compared to CpTi, Ti6Al4V, and Ti3Al2V. It was observed that the presence of Cu in the Ti3Al2V matrix allowed for a cathodic shift in OCP within the depassivation (tribological testing) and re-passivation domain.

### Histological Analysis-Bone-Implant Interface

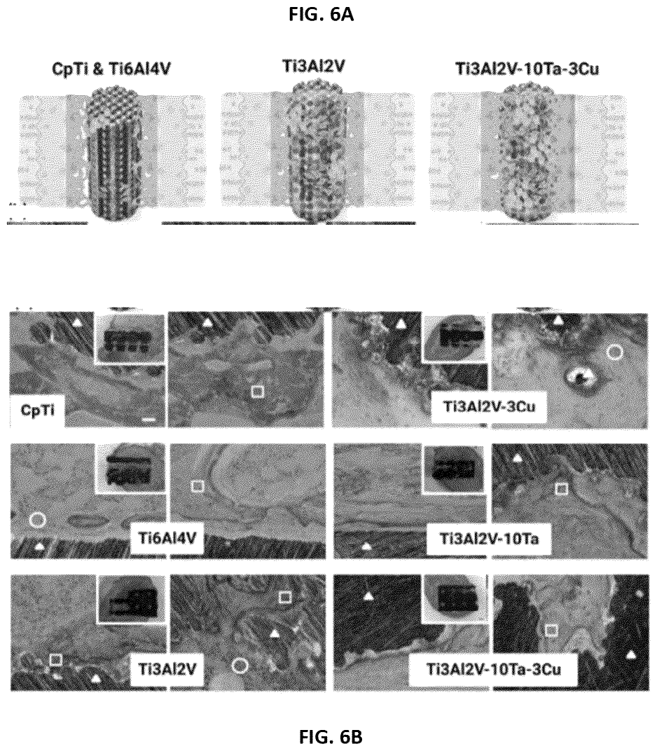

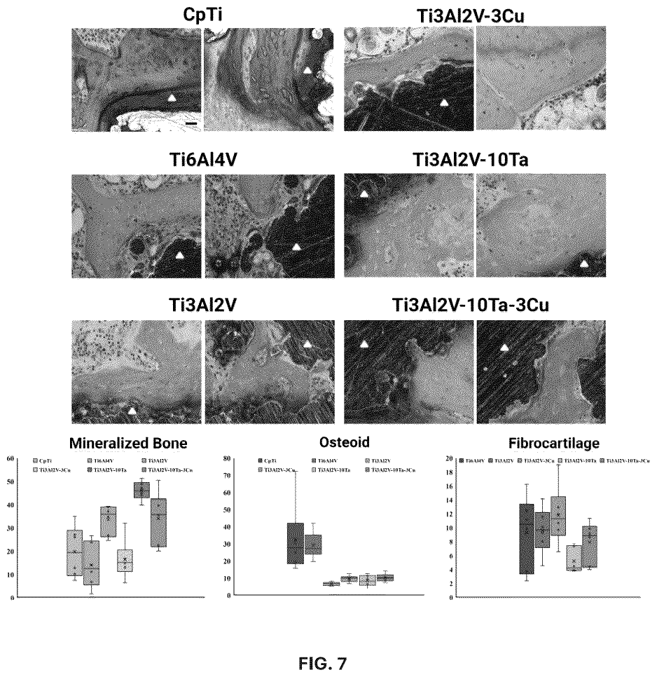

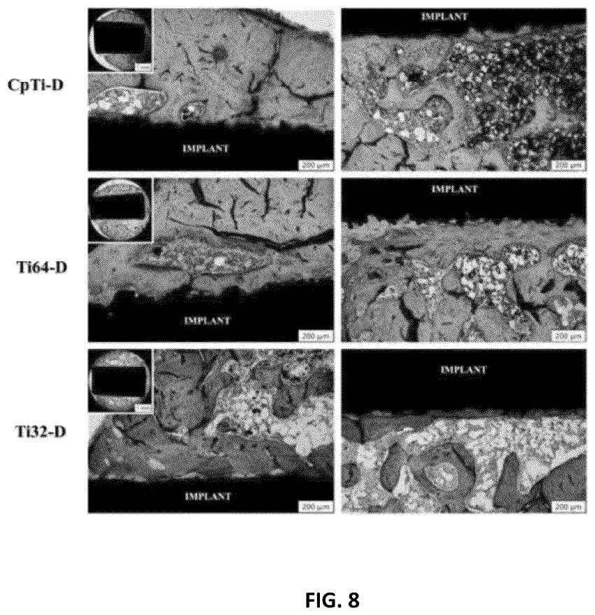

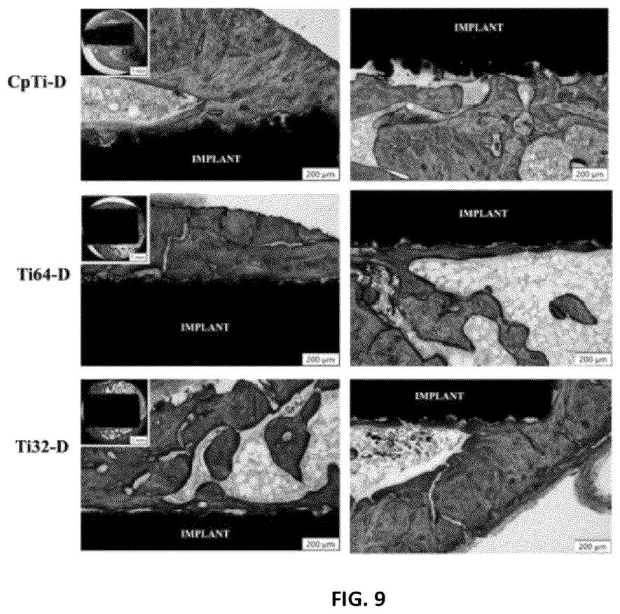

In vivo rat model with CpTi as the positive and Ti6Al4V as the negative control was studied with dense and porous implants, FIG. 6, FIG. 7, FIG. 1B, FIG. 8, and FIG. 9, show SRBS and H&E-stained bone sections for dense implants, which were primarily considered an in vivo control for comparison. The H&E and SRBS-stained bone sections for porous implants are presented in FIG. 6 and FIG. 7, showing pore cross-sections from different layers for each composition.

FIG. 7 in particular, shows Sanderson's Rapid Bone-Stained micrographs (SRBS). Areas (shown as Reddish/orange) represent matured trabecular bone, while darker and lighter (shown as blue) areas represent osteoid tissue or a combination of fibrocartilage with osteoid tissue, respectively. Dark ((shown as blue rounded dots) are osteoblasts recruited in the newly formed osteoid tissue at the bone integration front. Some areas of a (shown as lighter blue) with more elongated cells represent chondrocyte cells in the fibrocartilage. Ti3Al2V clearly shows bone apposition and osseointegration at the BIC compared to clear gaps for CpTi and Ti6Al4V. The presence of well-embedded osteocytes in the trabecular area for Ti6Al4V composition at 4 weeks post-implantation suggests older bone that could be a residual continuation from the outer cortical area. Adding Cu to Ti3A13V-3Cu composition does not elicit any inflammation or neoplasia, suggesting non-toxicity; however, there is evidence of the delayed onset of osseointegration compared to Ti3Al2V composition alone. Positive control Ti3Al2V-10Ta shows very well-integrated trabecular bone at the BIC with trabecular bone width higher than the rest of the compositions. Interestingly, adding 10% Ta to Ti3A13V-3Cu reverses the delayed onset of osseointegration and shows the overall best performance among all six compositions. Graphs present a quantitative analysis of the observations made from SRBS-stained histology sections based on ImageJ Trainable Weka Segmentation. The scale bar in the first image represents 25 μm and is uniform for all images. Statistical information from Tukey-Kramer pairwise comparisons is presented in Supplemental Tables 2-4 as follows.

TABLE 2
Statistical Tukey-Kramer pairwise comparison between all six
in vivo compositions for area fraction of mineralized bone

| | Mineralized Bone | |
|---|---|---|
| | Pairwise-Tukey Comparison | Difference in means |
| 1 | μTi6Al4V-μCpTi | Not Significant |
| 2 | μTi3Al2V-μCpTi | Not Significant |
| 3 | μTi3Al2V-μTi6Al4V | Significant |
| 4 | μTi3Al2V-3Cu-μCpTi | Not Significant |
| 5 | μTi3Al2V-3Cu-μTi6μAl4V | Not Significant |
| 6 | μTi3Al2V-3Cu-μTi3Al2V | Significant |
| 7 | μTi3Al2V-10Ta-μCpTi | Significant |
| 8 | μTi3Al2V-10Ta-μTi6Al4V | Significant |
| 9 | μTi3Al2V-10Ta-μTi3Al2V | Not Significant |
| 10 | μTi3Al2V-10Ta-μTi3Al2V-3Cu | Significant |
| 11 | μTi3Al2V-10Ta-3Cu-μCpTi | Not Significant |
| 12 | μTi3Al2V-10Ta-3Cu-μTi6Al4V | Significant |
| 13 | μTi3Al2V-10Ta-3Cu-μTi3Al2V | Not Significant |
| 14 | μTi3Al2V-10Ta-3Cu-μTi3Al2V-3Cu | Significant |
| 15 | μTi3Al2V-10Ta-3Cu-μTi3Al2V-10Ta | Not Significant |

TABLE 3
Statistical Tukey-Kramer pairwise comparison between all six
in vivo compositions for area fraction of osteoid tissue.

| | | |
|---|---|---|
| Pairwise-Tukey Comparison | Osteoid | Difference in means |

| | | |
|---|---|---|
| 1 | μTi6Al4V-μCpTi | Not Significant |
| 2 | μTi3Al2V-μCpTi | Significant |
| 3 | μTi3Al2V-μTi6Al4V | Significant |
| 4 | μTi3Al2V-3Cu-μCpTi | Significant |
| 5 | μTi3Al2V-3Cu-μTi6Al4V | Significant |
| 6 | μTi3Al2V-3Cu-μTi3Al2V | Not Significant |
| 7 | μTi3Al2V-10Ta-μCpTi | Significant |
| 8 | μTi3Al2V-10Ta-μTi6Al4V | Significant |
| 9 | μTi3Al2V-10Ta-μTi3Al2V | Not Significant |
| 10 | μTi3Al2V-10Ta-μTi3Al2V-3Cu | Not Significant |
| 11 | μTi3Al2V-10Ta-3Cu-μCpTi | Significant |
| 12 | μTi3Al2V-10Ta-3Cu-μTi6Al4V | Significant |
| 13 | μTi3Al2V-10Ta-3Cu-μTi3Al2V | Not Significant |
| 14 | μTi3Al2V-10Ta-3Cu-μTi3Al2V-3Cu | Not Significant |
| 15 | μTi3Al2V-10Ta-3Cu-μTi3Al2V-10Ta | Not Significant |

TABLE 4
Statistical Tukey-Kramer pairwise comparison between all six
in vivo compositions for area fraction of fibrocartilage.

| | | Fibrocartilage |
|---|---|---|
| | Pairwise-Tukey Comparison | Difference in means |
| 1 | μTi3Al2V-μTi6Al4V | Not Significant |
| 2 | μTi3Al2V-3Cu-μTi6Al4V | Not Significant |
| 3 | μTi3Al2V-3Cu-μTi3Al2V | Not Significant |
| 4 | μTi3Al2V-10Ta-μTi6Al4V | Not Significant |
| 5 | μTi3Al2V-10Ta-μTi3Al2V | Not Significant |
| 6 | μTi3Al2V-10Ta-μTi3Al2V-3Cu | Significant |
| 7 | μTi3Al2V-10Ta-3Cu-μTi6Al4V | Not Significant |
| 8 | μTi3Al2V-10Ta-3Cu-μTi3Al2V | Not Significant |
| 9 | μTi3Al2V-10Ta-3Cu-μTi3Al2V-3Cu | Not Significant |
| 10 | μTi3Al2V-10Ta-3Cu-μTi3Al2V-10Ta | Not Significant |

The H&E staining for the bone-implant sections was primarily evaluated for in vivo inflammatory response towards Ti3Al2V, Ti3Al2V-3Cu, and Ti3Al2V-10Ta-3Cu chemical makeup as an implant material. None of the three material compositions show inflammatory markers or responses, including necrosis and neoplasia. Even though H&E histology micrographs are represented in varying shades of pink/purple, visible demarcations are present for all compositions, which aids in determining their relative biological responses. The SRBS histology micrographs for dense and porous implants show qualitative evidence of new bone or osteoid formation, osteoblast recruitment, and bone maturation or trabecular bone formation in the region of interest across all compositions except for Ti6Al4V. However, detailed analysis suggests gaps at the bone-implant contact (BIC) surface with multiple focal fibrocartilaginous areas at BIC for CpTi. Markedly, there was no significant bone ingrowth into the pores in CpTi. Some areas show interwoven lamellar bone into the fibrocartilage in Ti3Al2V. The lighter blue stain indicates the onset of fibrocartilage with more flattened and organized elongated cell rows. In comparison, Ti6Al4V shows almost 90% of the interface area covered with fibrous tissue (dark blue), including pore infiltration with isolated areas of osteoid presence. Some areas show old/matured cortical bone segments from surgical procedures. Fibrocartilage presence at a distance from the implant shows inferior osseointegration affinity towards the implant material. On the other hand, Ti3Al2V shows no gaps at the BIC with very well-apposed osteoid tissue (reddish orange) and continuing osteoblast recruitment. Bone tissue infiltration takes precedence over fibrocartilage into the porous channels of the bulk implant.

Ti3Al2V-3Cu alloys show similar characteristics of bone remodeling to CpTi implants with osseous tissue at the BIC and interwoven lamellar bone into the implant surface. The fibrocartilage that runs through the porous channel has focally embedded new bone tissue. Ti3Al2V-10Ta shows enhanced bone ingrowth into the implant's pores and osteoid lining fronts, suggesting continued bone remodeling. However, the fibrocartilaginous presence in Ti3Al2V-3Cu alloys is shown to be restored to the bony formation and new osteoid tissue inside the pores of the implant in Ti3Al2V-10Ta-3Cu compositions with the addition of 10% Ta.

Quantitative histomorphometry (Table 5 shown below in combination with FIG. 7) provides a clearer idea about the remodeling process undergone for all six compositions at 6 weeks post-implantation.

TABLE 5
Showing scored parameters for bone regeneration around
BIC based on scoring criteria mentioned earlier.

| | | | | | | Ti3Al2V- |
|---|---|---|---|---|---|---|
| | CpTi | Ti6Al4V | Ti3Al2V | Ti3Al2V-3Cu | Ti3Al2V-10Ta | 10Ta-3Cu |
| Trabecular | 1 | 1 | 2 | 1 | 2 | 2 |
| apposition | | | | | | |
| Fibrocartilage | 0 | 1 | 1 | 1 | 1 | 1 |
| presence | | | | | | |
| Osteoid at the | 1 | 1 | 3 | 2 | 4 | 3 |
| interface | | | | | | |
| Inflammation | 0 | 0 | 0 | 0 | 0 | 0 |
| Fibrosis | 0 | 2 | 0 | 1 | 0 | 0 |
| Tissue | 1 | 0 | 3 | 1 | 4 | 4 |
| ingrowth into | | | | | | |
| the device | | | | | | |

First, the numbers show a uniform fibrocartilage presence across all the compositions, whether visibly at the BIC extended from the lateral edge of the implant exposed to multiple different tissues apart from the osseous phenotype. Such fibrocartilage response is expected at an early timeframe, like 6 weeks, which then develops into woven bone. However, there is only a significant difference in fibrocartilage presence in Ti3Al2V-3Cu (11.8±4) and Ti3Al2V-10Ta (5.2±2) due to much higher trabecular bone presence for Ti3Al2V-10Ta (46±3) compared to other compositions.

Comparing the box plots for mineralized bone and osteoid presence at the BIC (see FIG. 7), there is a significant change in bone maturation between CpTi, Ti6Al4V, and the rest of the compositions, indicating enhanced biocompatibility and biological response of native tissue towards the implant material. Overall, Ti6Al4V shows the least amount of mineralized bone (14±8), which is not significantly different from that of CpTi (19.7±10), which corresponds to the higher values for osteoid presence at the BIC for these compositions 29±7 and 32.4±19 respectively. However, with changing the chemical composition of the alloy to Ti3Al2V, a significant jump in native tissue response is observed with the fraction of mineralized bone area 34±5 compared to Ti6Al4V. This jump is reflected in the lower area fraction of osteoid in Ti3Al2V (6.7±1). With the addition of 3% Cu to the modified alloy Ti3Al2V, although we notice a significant change from Ti3Al2V, the area fraction of mineralized bone does not significantly change from that of CpTi. Therefore, it is safe to deduce that bone maturation might be delayed due to Cu addition; biocompatibility is not compromised. Since Ti3Al2V-10Ta (positive control) shows the highest amount of mineralized bone (46±3), adding 10% Ta to Ti3Al2V-3Cu alloys speeds up the delayed bone remodeling at the same level as Ti3Al2V-10Ta as well as Ti3Al2V alloys owing to non-significant statistical differences.

### Infection Prevention and Bacterial Resistance

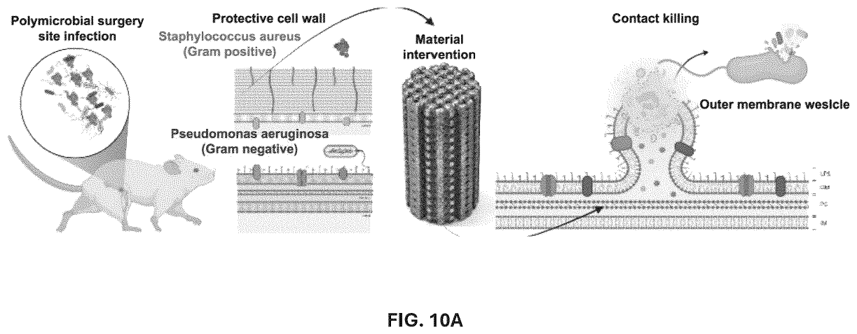

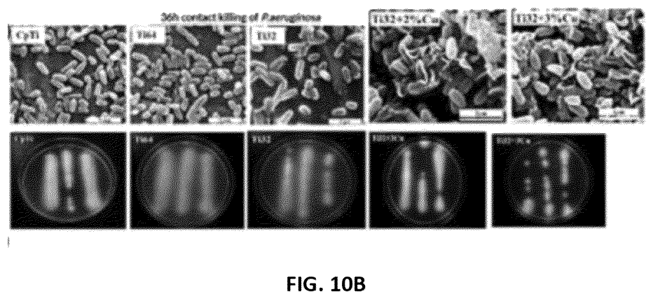

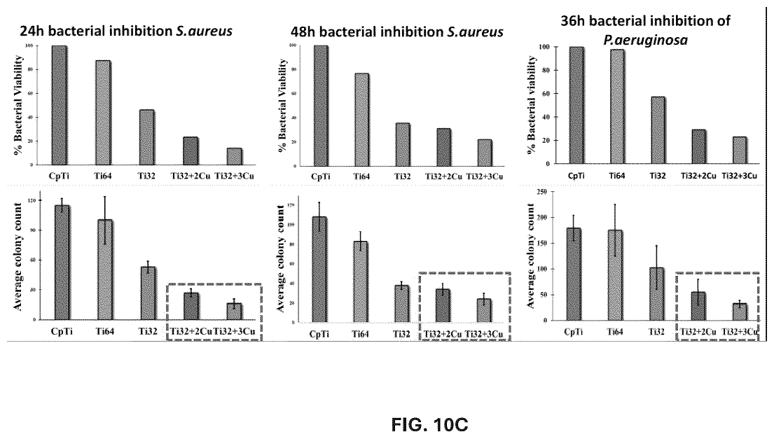

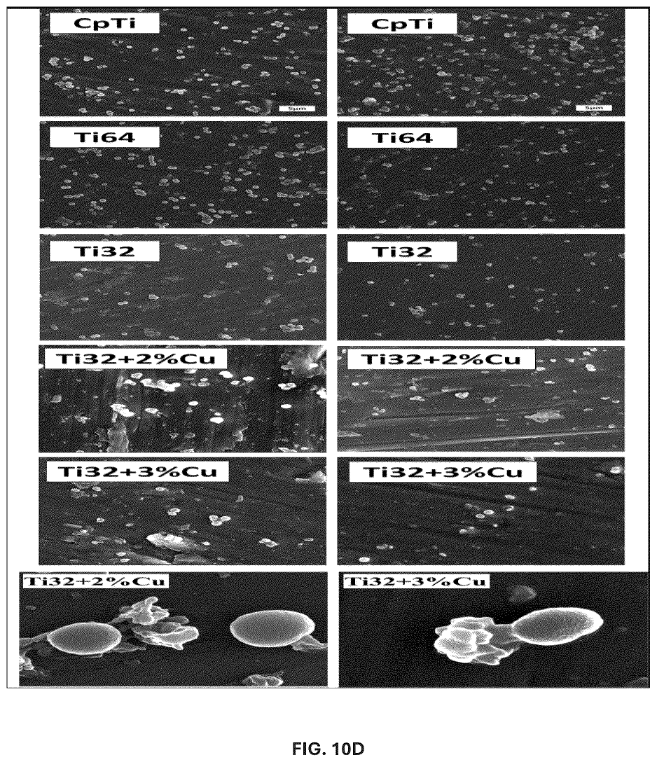

Both CpTi and Ti6Al4V do not possess inherent antibacterial resistance [32], [33]; therefore, we do not expect any antibacterial properties in Ti3Al2V by extension. However, since post-surgical implant site infection is a common denominator in orthopedic material (see FIG. 10A), examining currently used orthopedic materials is not beyond the scope of this research. As a confirmatory test, bacterial viability across all compositions was tested for 36 h with P. aeruginosa (gram-negative) colonies. Cetrimide (Pseudosel agar) is a selective medium to culture this bacterium. SEM micrographs (see FIG. 10B) also corroborate a reduction in planktonic P. aeruginosa bacterial cells on the surface of Ti3Al2V compared to that of CpTi and Ti6Al4V. However, bacterial inhibition was significantly enhanced after 36 h of culture for Ti3Al2V-2Cu and Ti3Al2V-3Cu compositions, showing deflated bacterial cells from cytoplasmic outflow (in red). P. aeruginosa secretes a pigment pyoverdine that emits fluorescent green color under UV light on cetrimide agar showing significantly lesser colony formation on agar plates for Ti3Al2V-2Cu and Ti3Al2V-3Cu, (see FIG. 10C) % Bacterial viability evaluated for all compositions show the antibacterial efficacy of Ti3Al2V-2Cu and 3Cu alloys, which were 70% and 80% higher than CpTi after 36 h of culture, respectively. The antibacterial efficacy of CpTi and Ti6Al4V was observed to be within error margins of each other and under 4% since neither showed any antibacterial properties. However, for Ti3Al2V, the reduced live bacteria on the surface by 57% was a marked and novel identification of material properties for Ti3Al2V alloys.

This data was beneficial for establishing Ti3Al2V—Cu alloys as a material for implant applications. A secondary bacterial culture with a different strain of bacteria S. aureus (gram-positive), for 24 and 48 h on all compositions was performed to confirm the antibacterial efficacy of the alloys herein. Bacterial cells were counted in triplicate from SEM micrographs/unit area based on magnification instead of the agar plate method. % Living bacteria for Ti3Al2V-2Cu and 3Cu compared to CpTi at 24 h of culture were 23 and 14%, respectively. However, at 48 h there was a slight but insignificant increase in the bacterial count for both compositions. A similar result as with the previous bacteria was observed for Ti3Al2V, the % bacterial viability was significantly lower than CpTi and 46 and 36% after 24 and 48 h of culture, respectively. According to the SEM micrographs shown in (see FIG. 10D), significantly lower S. aureus bacterial cells can be observed in SEM images on the surface of Ti3Al2V compared to that on CpTi and Ti6Al4V for both time points. A ruptured cell wall is seen and disruption of the cell membrane for S. aureus bacterium on Ti3Al2V-2Cu and 3Cu.

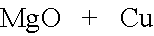

MgO and Cu were incorporated into CpTi matrix to enhance its osteogenic potential and imbue inherent bactericidal capabilities.

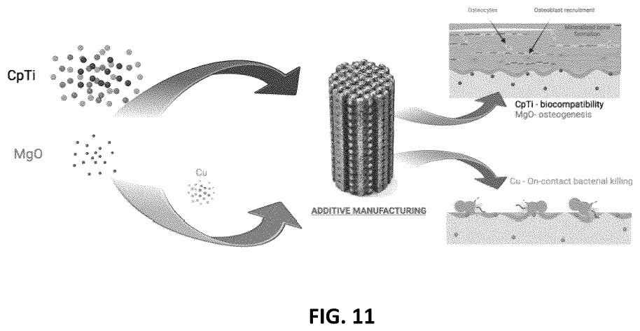

This Ti—MgO—Cu material chemistry is expected to enhance early-stage osseointegration due to osteogenic MgO and prevent polymicrobial infection incidence at the implant site, ensuring the implant's long-term stability and preventing the need for revision procedures due to their aseptic loosening. This study aims to fabricate CpTi, CpTi+1 wt. % MgO (CpTi—MgO), and CpTi+1 wt. % MgO+3 wt. % Cu (CpTi—MgO—Cu) compositions using metal-AM. These compositions were characterized for microstructure and microhardness evaluations. In vivo rat studies were conducted to evaluate the biological performance of these compositions. Structures utilized for the in vivo studies were ˜40 vol. % porous with an approximate pore size of 600-700 μm since pore sizes in this range are optimum for enhanced tissue integration and osseointegration. Additionally, in vitro bacterial culture was studied using the commonly occurring Staphylococcus aureus bacterial strain to evaluate the antibacterial efficacy of CpTi-MgO—Cu. We hypothesize that the CpTi—MgO—Cu composition will demonstrate better osseointegration performance than CpTi in vivo with no cytotoxicity due to the presence of Cu, as schematically shown in FIG. 11.

### Materials and Methods

### Processing of Samples Using Metal Additive Manufacturing

CpTi, CpTi—MgO, and CpTi—MgO—Cu compositions were processed using metal additive manufacturing. A metal matrix composition of CpTi—MgO was prepared by premixing CpTi powders (GKN Hoeganaes, Cinnaminson, NJ) with 1 wt. % of MgO (Inframat® Advanced Materials™, Manchester, CT) powders. Similarly, CpTi—MgO—Cu composition was prepared by premixing CpTi powders with 1 and 3 wt. % s of MgO and Cu (GKN Hoeganaes, Cinnaminson, NJ) powders, respectively. All metal and ceramic powders used for fabrication were sieved to obtain a powder particle size of <63 μm. All metal and ceramic powers used were spherical. The fabrication used two AM processes: directed energy deposition (DED) and selective laser melting (SLM). Samples for in vitro study were printed on a 5-axis DED-based AM system (FormAlloy, Spring Valley, CA). Although coarser powder particles (45-150 μm) are preferred for DED-based AM systems, we have optimized the print parameters to accommodate finer particle sizes of <63 μm for the printing operation. The printing operation was conducted in an argon-purged environment with O2<20 ppm in the print chamber. A cold rolled CpTi substrate was used as a build plate. Discs of 8 mm diameter and 4 mm height were printed on the DED system. The printing parameters used for the compositions are presented in Table 1. Samples used for in vivo study were printed on an SLM-based powder bed fusion system (3D Systems ProXR DMP 200, Rock Hill, SC) with a 300 W fiber laser and wavelength 2=1070 nm. Porous structures of 2.4 mm diameter and 4 mm height with ˜40 vol. % porosity were designed in 3DXpert CAD Software (3D Systems, Rock Hill, SC). Premixed powders were poured into the supply chamber and compacted using a companion plate. A thick CpTi plate of ˜2.5 thickness was used as the build platform and placed-secured on the melting stage. A roller system carried powders from the supply to the build stage, with 30 μm as the layer thickness. The laser power and scanning speed for all the compositions are reported in Table 1. 3D Systems provide printing parameters used for CpTi and CpTi—MgO as the standard Ti print parameters. Laser power has been increased by 10% and scan speed reduced by 10% to increase the print energy input for CpTi—MgO—Cu since Cu displays poor laser absorption and needs higher energy for additive operation. Porous cylinders post-printing were cut from the build plate and subjected to repeated sonication in de-ionized (DI) water and ethanol, followed by compressed air treatment to remove any loose powder particles inside the pores. The final step in residual powder removal involved acid etching in 1% H.F. in DI water. The samples were sonicated again in DI water and ethanol to remove any acid residues.

TABLE 6
Print-processing parameters used for DED and PBF operations of additively
manufactured CpTi, CpTi—MgO, and CpTi—MgO—Cu compositions.
DED (in vitro, microstructure, hardness)

| | Laser | | Scan speed | | | | | |
|---|---|---|---|---|---|---|---|---|
| | power (W) | | (mm/min) | | Shield gas | Carrier gas | Powder disc | Slice |
| Composition | Contour | Hatch | Contour | Hatch | (l/min) | (l/min) | speed (rpm) | (mm) |
| CpTi | 350 | 350 | 1500 | 1200 | 18 | 14 | 0.7 | 0.3 |
| CpTi—MgO | | | | | | | | |
| CpTi—MgO—Cu | | | | | | | | |

PBF (in vivo ~40% porosity)

| | Laser | Scan | |
|---|---|---|---|
| | power | speed | Slice |
| Composition | (W) | (mm/s) | (μm) |
| CpTi | 180 | 1600 | 30 |
| CpTi—MgO | | | |
| CpTi—MgO—Cu | 198 | 1440 | |

### Microhardness and Microstructure

DED printed discs were cut off the build plate and subjected to grinding on SiC grinding papers, 80-2000 grit size. This was followed by alumina suspension polishing, reducing the alumina powder particle size from 1-0.05 μm. Vickers microhardness test was conducted on a Phase II Plus Micro Vickers Hardness tester (Upper Saddle River, NJ, USA) using a load of 200 gms and a dwell time of 15 s. Hardness values on a polished surface perpendicular to the build direction were obtained. An n=5 measurement was taken for each point. For acquiring the microstructures, polished surfaces of the discs were etched in Kroll's reagent for 45 s and observed under a Scanning Electron Microscope (SEM, Apreo, Thermo Scientific, MA, USA).

In Vitro Bacterial Study—Staphylococcus aureus

Bacterial culture was carried out on CpTi and CpTi—MgO—Cu to evaluate the antibacterial resistance using Staphylococcus aureus gram-positive bacterial strain for 24, 48, and 72 h. Freeze-dried S. aureus (Carolina Biological, NC) was rehydrated using rehydration media. Tryptic soy broth was used as the nutrient medium. The rehydrated bacterium was subjected to nutrient broth dilutions to obtain 0.5 McFarland standard optical density measurement corresponding to 106 CFU/ml of bacteria. Polished disc samples were sterilized before culture, placed in 24 well plates, and studied in triplicate for agar plate colony count and duplicates for SEM characterization. 106 CFU/ml of bacterial colonies were seeded on the surface of the discs, with 2 ml of tryptic soy broth added as the nutrient medium in each well. After the respective time points, bacterial cells from triplicate samples for agar plate colony count were scraped using cell scrapers and mixed in 2 ml of 0.1 M PBS and serially diluted to approximately contain 10 to 100 colonies in 1 μl of the solution. 1 μl of this solution was streaked on a tryptic soy agar plate and incubated for 24 hrs. The duplicate samples used to observe the bacterial cell morphology were subjected to a fixative solution, dehydrated as described in Section 2.4, and observed under a Scanning Electron Microscope (Quanta 200F, Thermo Fisher, Waltham, USA). Images were taken at 300× for each composition, and the number of bacterial cells was counted on at least n=4 images for each composition. The antibacterial efficacy for agar plate count at 24 hrs was evaluated as a function of bacterial colonies counted on individual material compositions, as

$$N = C \times d \times 10 \; 00 / l$$

$$R = ( {N}_{control} - {N}_{material} ) / {N}_{control} \times 100 \% ,$$

Where N is the calculated number of bacterial colonies observed, C is the average colony count on a plate, d=dilution factor, and l=volume of bacterial suspension on the sample. Antibacterial efficacy from SEM images was evaluated for 24, 48, and 72 h time points and calculated for R, with N being the average number of bacterial cells from multiple SEM images.

### In Vivo Study

CpTi, CpTi—MgO, and CpTi—MgO—Cu compositions were subjected to an in vivo rat study. CpTi is known to demonstrate excellent in vivo biological performance and is used as a control. Adding MgO to CpTi is expected to show better early-stage osseointegration performance than CpTi. PBF-fabricated porous implants with ˜40 vol. % porosity were used for the study.

### Surgery and Implantation Procedure

Male Sprague-Dawley rats with average weights between 300-350 gms were used for the in vivo study. Post procurement, the animals were acclimatized in temperature and humidity-controlled rooms in separate cages for at least two weeks. Buprenorphine (0.3 mg/kg) for alleviating pain was subcutaneously administered to the animals 30 mins before anesthesia. A prescribed dose of IsoFlo® (isoflurane, USP, Abbott Laboratories, North Chicago, IL, USA) coupled with oxygen (Oxygen USP, A-L Compressed Gases Inc., Spokane, WA, USA) was used to anesthetize the animals. Once the animal's movements ceased under anesthesia, the implantation area around the femur and the knee was shaved thoroughly and cleaned thrice with chlorohexidine and isopropyl alcohol scrubs. 0.3 ml of Lidocaine HCL (without epinephrine), 0.5% as a localized numbing agent, was subcutaneously administered near the implantation area on each leg. The animal was transferred onto a sterile surgery table area. A 2-inch incision was made along the femur on the lateral side above the distal femoral condyle. A unicortical defect of 2.4 mm diameter was made on the lateral epicondyle using gradually increasing drill bits and rinsed with saline to prevent thermal necrosis and remove bone fragments. The implant was placed, the fascia was over the incision, and the skin was sutured with undyed braided coated MONOCRYL-polyglactin 910 (Ethicon Inc., Somerville, NJ, USA). The incision area was cleaned with saline scrubs and stapled. A similar procedure was carried out on the other leg of the animal. The animal was periodically monitored by respiration rate during the surgery procedure. Lactated ringers solution (LRS, 3 ml) for rehydration was subcutaneously administered to the animal post-surgery, followed by meloxicam (0.2 mg/kg) administration as an anti-inflammatory analgesic, and monitored until the animal regained consciousness. Postoperative care was carried out for 3 days, with buprenorphine administration every 12 hours and meloxicam every 24 hours. The animals were euthanized after 6 weeks of surgery by carbon dioxide overdose, followed by cervical dislocation as a secondary measure, and the femoral bone with the metal implant was harvested. The Institutional Animal Care and Use Committee (IACUC) of Washington State University (WSU-Pullman, WA) approved protocol was followed to perform the experimental and surgical procedure.

### Histological and Histomorphometric Analysis

The bone-metal explants were fixed in 10% neutral buffered formalin for at least 72 hrs for tissue infiltration. Serial dehydration in ethanol followed by embedment in polymethyl methacrylate (PMMA). These embedded bone explants were cut on Ekakt™ saw into 200 μm thin sections, mounted on glass slides, and then ground to 20-50 μm thick sections using 1200 grit size sanding paper on Ekakt 400 micro grinder. The sections were then polished on the micro grinder using 4000 grit-size paper. Gomori trichrome, Hematoxylin & Eosin (H&E), and Sanderson's Rapid Bone Staining (SRBS) were the stains on separate bone sections for each composition. The stained bone sections were imaged on a Keyance digital microscope (Model VHX-7000, Itasca, IL). H&E-stained slides were imaged for any visible markers indicating an inflammatory response in areas around the implant. Gomori trichrome-stained slides were observed for muscle fibers and collagen presence at the bone-implant contact (BIC), and SRBS-stained slides for mineralized bone formation, osteoid presence at the BIC, and mineralization fronts.

Histomorphometric analysis was carried out using SRBS-stained slides for each composition. To restrict the region of interest (ROI) to 100-150 μm from the implant surface, images were captured at 1000× magnification around the bone-implant contact (BIC) region. Quantitative evaluation of mineralized bone formation at the BIC was carried out using Trainable Weka Segmentation in ImageJ with Random Forest Algorithm for individual images. At least 7 regions were analyzed to quantify mineralized bone formation at the BIC and presented in % area fraction.

### Results

CpTi shows good biocompatibility and no cytotoxicity. Its lack of strength and fatigue resistance over Ti6Al4V makes it a popular coating material choice over bulk Ti6Al4V implants. However, it is bio-inert and possesses no antibacterial capabilities. The surface properties of the implant influence its biological performance in the physiological environment. This study aims to enhance the early-stage osseointegration of CpTi with MgO addition and induce inherent antibacterial capabilities by adding Cu. This CpTi—MgO—Cu material chemistry is expected to show superior biological performance in vivo compared to CpTi and can be a potential metallic coating material of choice on bulk metallic implants.

### Microstructure and Hardness

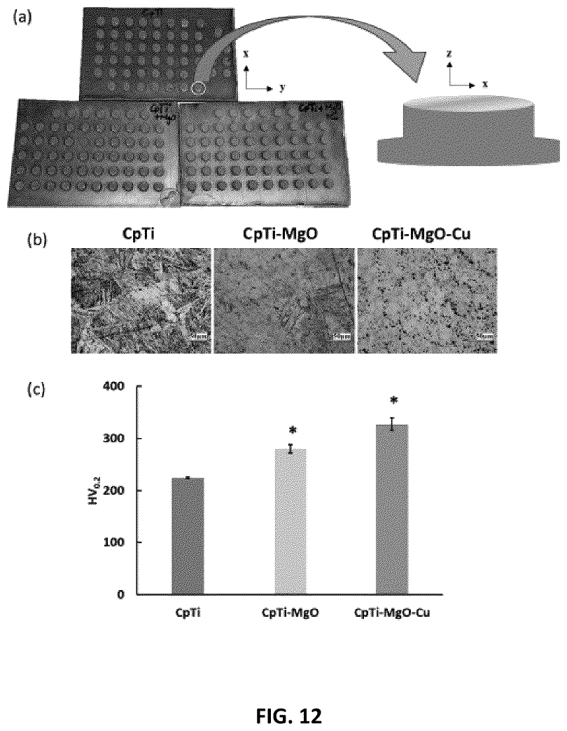

SEM micrographs of the etched surface, FIG. 12b, for CpTi and CpTi—MgO, show typical α′ martensitic needle-like structures typically observed in additively manufactured CpTi due to the fast-cooling nature of the process. With Cu addition, we observe keyhole porosities owing to balling effect and splashing of molten material. Cu has a high thermal diffusivity, almost 100 times that of Ti, and laser absorption of Cu is very poor. With the high viscosity of Cu in the melt-pool compared to that of Ti, a shallower and wider melt-pool is created by splashing molten particles leading to keyhole porosities.

Vickers microhardness measurements conducted on the polished surface of the DED printed compositions, FIG. 12c, revealed a hardness of 224±2 HV0.2 on CpTi surface, similar to those observed in previous work. With MgO addition in CpTi, the microhardness value increased to 280±8 HV0.2 due to reinforced MgO particles in the CpTi matrix providing resistance to deformation. Cu addition in CpTi—MgO increased the hardness to 327±12 HV0.2 due to Ti2Cu intermetallic formation and solute solution strengthening by Cu solute atoms.

### Histological Analysis and Histomorphometry—Bone-Implant Interface

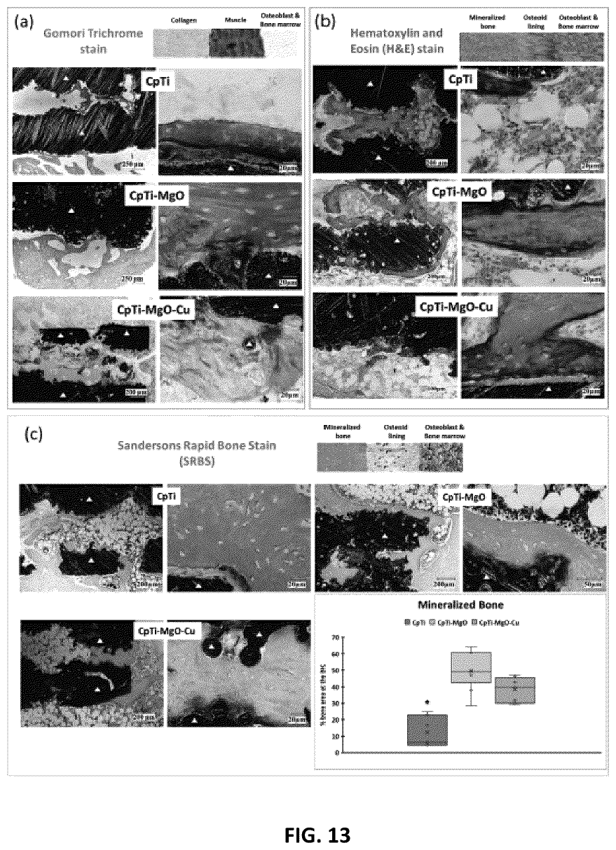

To assess the biological performance of the compositions in a physiological environment, an in vivo rat model was utilized. CpTi was considered the control with CpTi—MgO and CpTi—MgO—Cu as the treatment compositions. FIG. 13 presents Gomori Trichrome, Hematoxylin & Eosin (H&E), and Sanderson's Rapid Bone Stained (SRBS) bone sections of porous implants with approximately 40 vol. % porosity. H&E-stained bone sections, FIG. 13b, were observed for possible inflammatory markers. None of the compositions show any inflammatory response, including neoplasia and necrosis. Gomori Trichrome stain was employed to evaluate muscle fiber formation and collagen presence at the bone-implant contact (BIC), FIG. 13a. At the BIC, all three compositions exhibited interwoven muscle fibers within collagenous regions, indicating early-stage osseointegration and mineralization front. Notably, these regions appeared visibly thinner for CpTi than CpTi—MgO and CpTi—MgO—Cu, suggesting a higher degree of osteogenesis in the latter compositions attributed to MgO. Further examination at higher magnification revealed gaps at the BIC for CpTi, whereas, for CpTi—MgO and CpTi-MgO—Cu, these gaps were filled with osteogenic fronts accompanied by small areas of muscle fiber presence. Compared to CpTi—MgO and CpTi—MgO—Cu, the pink-bluish regions at the BIC for CpTi—MgO indicated the presence of muscle fibers interwoven with collagenous regions. Conversely, the bright pink regions at the BIC for CpTi—MgO—Cu indicated the presence of muscle fibers without collagen, suggesting delayed bone maturation and osseointegration due to the presence of Cu.

The observations described above are further supported by the H&E-stained histology micrographs, FIG. 13b, where varying shades of pink and purples reveal distinct demarcations representing mineralized bone, osteoid lining, and osteoblast recruitment regions. In the case of CpTi, only certain regions exhibit a mineralized bone front at the BIC, while the rest show its absence. In contrast. CpTi—MgO visibly demonstrates a higher presence of mineralized bone directly at the BIC, followed by an osteoid mineralization front, indicating superior early-stage osteogenic performance. Upon closer examination at the BIC, CpTi—MgO reveals the infiltration of mineralization fronts into the implant area, encompassing the implant regions. However, H&E histology for CpTi—MgO—Cu shows a lower degree of mineralized bone formation at the BIC compared to CpTi—MgO, although similar implant area infiltration features into the implant area are observed.

SRBS-stained histological micrographs, shown in FIG. 13c, depict trabecular bone formation, osteoid presence, and osteoblast recruitment at the bone-implant interface. Across all compositions, matured bone formation with well-embedded osteocytes and focal outward growth of osteoblastic regions from the implant surface can be observed. CpTi's trabecular bone formation is followed by a thick osteoid lining at the BIC. In contrast, both CpTi—MgO and CpTi—MgO—Cu compositions exhibit newly formed trabecular bone directly apposed to the outer surface of the implant, indicating superior osseointegration performance compared to CpTi. Furthermore, the SRBS-stained histology micrographs reveal a higher degree of matured bone infiltration in the porous channel for CpTi—MgO and CpTi—MgO—Cu than CpTi. To obtain a clearer idea on the remodeling process undergone at the BIC. SRBS-stained histology was employed for histomorphometric analysis to evaluate the matured bone formation at the BIC (FIG. 13c) within the specified ROI of 100 μm. Among the tested compositions, CpTi—MgO demonstrated the highest amount of matured bone formation at the BIC, 49.5±11.5%, followed by CpTi—MgO—Cu. 38.2±7.2%, while CpTi exhibited the least amount, 12.1±9.2%. This suggests a higher affinity and enhanced biological response of the host tissue toward the chemical makeup of CpTi—MgO and CpTi—MgO—Cu. Although the matured bone formation between CpTi—MgO and CpTi—MgO—Cu falls within the error range of each other, the difference in mean values can be attributed to the delayed osseointegration observed in the latter composition due to the presence of Cu, as discussed earlier.

### Infection Prevention

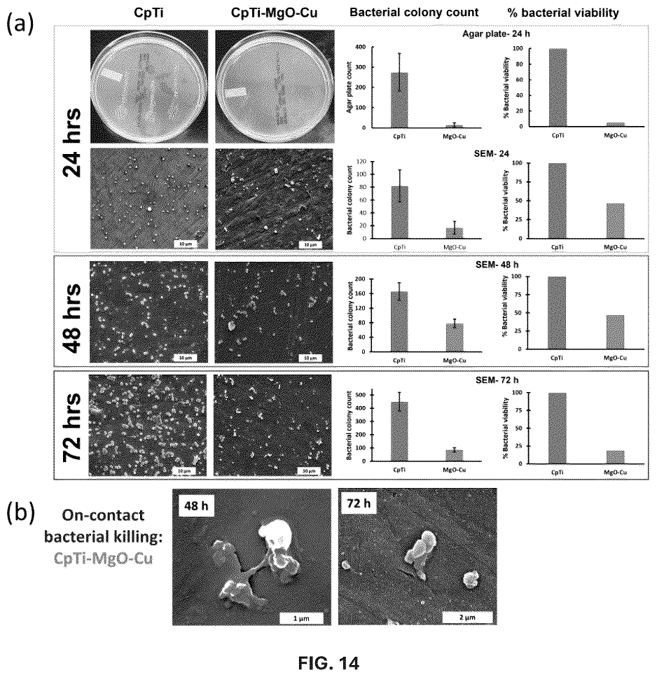

CpTi inherently does not possess antibacterial resistance. In order to address post-surgical infections, the addition of Cu was implemented in the CpTi—MgO composition. Cu is well-known for inhibiting bacterial growth through the on-contact killing of bacterial cells. Since Staphylococcus aureus is one of the most commonly occurring infections in vivo, the antibacterial efficacy of the CpTi—MgO—Cu material against this bacterial strain at 24, 48, and 72-h time points using CpTi as the negative control was evaluated, FIG. 14a. After 24 h of bacterial culture, we observed a significant reduction in bacterial viability on the agar plate for CpTi—MgO—Cu. Bacterial colony counting on the agar plate showed a 95% bacterial efficiency for CpTi—MgO—Cu compared to CpTi. Scanning electron microscopy (SEM) images taken after 24, 48, and 72 h of culture revealed a significant reduction in planktonic bacteria on the surface of CpTi—MgO—Cu. At the 24 h, the bacterial inhibition efficiency evaluated from the SEM images showed a 57% reduction in planktonic bacteria on the surface of CpTi—MgO—Cu. At 48 and 72 h, enhanced antibacterial efficiency was observed, with a 53% and 81% reduction in planktonic bacteria on the surface of CpTi—MgO—Cu. The SEM images at 48 and 72 h showed bacterial cells adhering to each other resulting in septum formation on the surface of CpTi-MgO—Cu, indicating evidence of cytoplasmic outflow leading to eventual disruption of the bacterial cell membrane. Higher magnification images (FIG. 14b) revealed the on-contact bacterial killing of S. aureus cells on the surface of CpTi—MgO—Cu, with ruptured cell walls and disruption of the cell membrane, resulting in cytoplasm outflow and eventual killing of bacterial cells at 48 and 72 h time points. CpTi—MgO—Cu demonstrated an excellent ability to inhibit infections at the end of the 72 h culture period.

The volume of orthopedic surgeries has been experiencing an exponential rise, with over 7 million orthopedic surgeries performed in the U.S. alone. According to a National Ambulatory Medical Care survey, 70% of patients visiting clinics for orthopedic surgery-related issues in 2015-2016 were over 45 years of age. Age plays a significant role in the quality of recovery, as the quality of bone and its healing ability significantly decreases with age. Elderly patients, who often suffer from immunocompromised bone health, experience prolonged recovery after surgery, compromising their overall health. Incidence of infections at the implant site requiring revision surgery, the impact on the patient's health is substantial, particularly for individuals with age-related degradation of bone health, leading to a further reduction in life expectancy. There is an unmet need in metallic implants for materials that can provide faster bone remodeling performance and infection prevention capabilities beyond what titanium currently offers. Current strategies include cemented implants with Ti6Al4V as the bulk material for strength with a surface coating of bioactive calcium phosphate (CaP) or hydroxyapatite (H.A.). Although cemented implants show superior in vivo performance towards early-stage osseointegration, one of the primary roadblocks includes delamination of the CaP coating due to poor metal-ceramic bonding. Instead, using porous titanium metallic coatings is a popular choice to abate the risk of coating failures. In order to induce osteogenic properties in these porous titanium coatings, MgO addition in CpTi can potentially solve the coating failure issue.

### Osteogenesis Due to MgO Addition

Mg plays an essential role in promoting bone calcification and remodeling. Mg deficiency in the bone has been linked with degenerative bone diseases such as osteoporosis. Mg regulates intracellular calcium ion concentration, pH, transporters, enzymes, and protein synthesis. Biodegradable Mg implants for low-load bearing bone-graft applications have been studied extensively. Moreover, incorporating MgO in calcium phosphate has enhanced cellular proliferation in vitro and osteogenic performance in vivo. In this study, incorporating MgO in CpTi enhanced osteogenesis at the bone-implant interface. With just 1 wt. % MgO addition in CpTi, mineralized bone formation at the BIC increased four-fold compared to that in CpTi, FIG. 13c. Bone remodeling at the implant surface follows osteoblast recruitment followed by osteoid lining and eventual maturation of bone. CpTi histology images show an osteoid lining at the bone-implant interface. For CpTi—MgO, histology images show mineralized bone directly apposed to the implant's outer surface, indicating an enhanced bone-remodeling process compared to CpTi. With superior osseointegration performance, we believe CpTi—MgO can potentially replace ceramic coatings in cemented implants and prevent coating failures.

### Antibacterial Performance Due to Cu Addition

The first recorded use of copper as a bactericidal date back 5000 years in Egyptian medical texts as a sterilizing agent for chest wounds. Having been used copper for medical purposes throughout the following generations, its antibacterial potential was realized in the 19th century. In 2011, copper was the first antimicrobial metallic material by the U.S. Environmental Protection Agency (EPA). Amid the current pandemic, in 2021, U.S. EPA announced and approved copper disinfecting products owing to the performance of copper and copper alloys against SARS-COV-2, i.e., the virus responsible for COVID-19. Realizing the potential of Cu as an antibacterial agent, extensive research has been conducted on incorporating Cu into Ti. Cu is a necessary trace element in the human body in a wide variety of tissues, but higher amounts of Cu can cause cytotoxicity leading to liver cirrhosis and neurologic abnormalities. A debate persists on the optimum amount of Cu in Ti. In this study, with 3 wt. % addition of Cu in CpTi, the H&E-stained bone sections show no signs of cytotoxicity. However, the mineralized bone formation in CpTi—MgO—Cu was observed to be lower than that in CpTi—MgO. Although 3 wt. % Cu did not cause cytotoxicity; there was a delayed early-stage osseointegration performance. CpTi—MgO—Cu still showed 3.5× mineralized bone formation at the interface than CpTi, showing superior osteogenic performance.

Early-stage osseointegration greatly affects the patient's recovery time. With CpTi—MgO—Cu used as a metallic coating on bulk Ti6Al4V alloy at load-bearing sites, coating failures in cemented implants can be avoided. At the same time, enhanced early-stage osseointegration can be achieved owing to the osteogenic properties of MgO, and inhibition of bacterial infections at the surgery site can prevent revision surgeries.

Early-stage osseointegration at the implant surface is critical in the post-surgery healing of elderly patients with degraded bone health. Ti's bio-inertness and non-antibacterial nature result in aseptic loosening and necessitate surgical intervention. Without adequate material intervention enhancing tissue integration and preventing polymicrobial infections, revision procedures further degrade the patient's health and potential patient morbidity. Ti6Al4V bulk implants coated with bioactive ceramics with osteogenic MgO and antibacterial Cu currently serve the purpose of coating failures due to weak metal-ceramic being a roadblock. Instead, we propose the addition of 1 wt. % MgO and 3 wt. % Cu in CpTi matrix as a coating on Ti6Al4V to prevent delamination failure owing to a strong Ti-on-Ti interface. In vivo studies showed superior osteogenic performance by CpTi—MgO and CpTi—MgO—Cu compositions. Histomorphometric evaluations reveal 4× enhanced mineralized bone formation in CpTi—MgO (49.5±11.5%) and 3.5× in CpTi—MgO—Cu (38.2±7.2%) in comparison to CpTi (12.1±9.2%) at the bone-implant interface. Additionally, no cytotoxicity was caused by 3 wt. % Cu addition. Antibacterial studies with commonly occurring Staphylococcus aureus bacterial stain showed up to 81% bactericidal effect by Cu in CpTi—MgO—Cu composition at the end of 72 hrs. A multifaceted metal-ceramic coating CpTi—MgO—Cu material makeup on bulk Ti6Al4V could serve as an ideal material for orthopedic implant applications with a reduction in implant failures and the need for revision surgeries due to delayed early-stage osseointegration and infection-related issues.

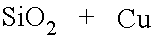

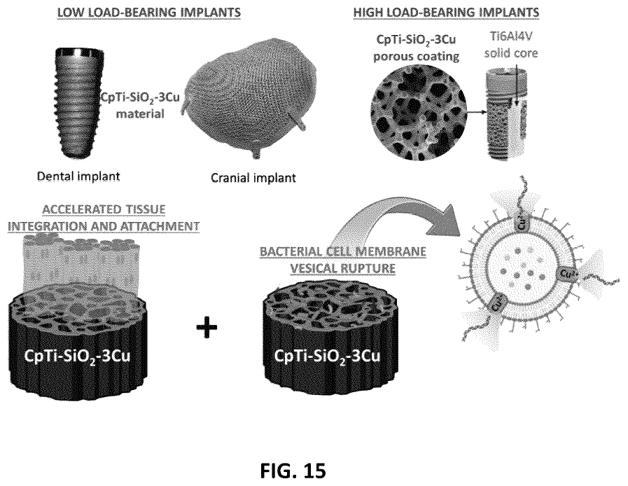

A multifaceted metal-ceramic system, CpTi—SiO₂-3Cu, was developed with enhanced early-stage osseointegration and bactericidal capabilities due to SiO₂ and Cu, respectively. The amount of SiO₂ was restricted to 1 wt. % to reduce the brittleness of the metal-ceramic systems. Considering the possibility of Cu toxicity, only 3 wt. % Cu was added. The compositional design (CpTi+1 wt. % SiO₂+3 wt. % Cu) coupled with the structural modification, i.e., porosities, has been implemented using AM. The compositions were tested for their biological performance and possible Cu cytotoxicity through in vivo rat studies. The antibacterial performance of CpTi—SiO₂-3Cu was investigated via in vitro bacterial culture with Staphylococcus aureus bacterial strain, a commonly occurring bacterial infection in the human body. CpTi—SiO₂-3Cu will demonstrate superior early-stage osseointegration to CpTi with inherent antibacterial capabilities. We anticipate that such composition can be used as a potential choice of monolithic material for low-load bearing implant applications and as a surface coating on Ti6Al4V for orthopedic devices (FIG. 15).

### Materials and Methods

### Additive Manufacturing of CpTi—SiO₂-3Cu

This study utilized CpTi, CpTi—SiO₂, and CpTi—SiO₂-3Cu compositions. To process the CpTi-SiO₂ metal matrix composition, CpTi powders (GKN Hoeganaes, Cinnaminson, NJ) were mixed with 1 wt. % of SiO₂ powder (Chemsavers Inc., Bluefield, VA). Similarly, the CpTi—SiO₂-3Cu composition was prepared by premixing CpTi powders with 1 wt. % of SiO₂ powders and 3 wt. % of Cu powders (GKN Hoeganaes, Cinnaminson, NJ). All metal and ceramic powders used in the additive manufacturing (AM) process were sieved to achieve a particle size<63 μm and possessed a spherical shape.

This study used directed energy deposition (DED) and selective laser melting (SLM) based AM processes to fabricate metal and metal-ceramic composite samples. Dense discs of ˜8 mm diameter and ˜4 mm height were printed on a 5-axis DED system (FormAlloy, Spring Valley, CA) for hardness, microstructure, and in vitro studies. Printing was conducted in an argon-purged environment within the print chamber, maintaining 02 levels below 20 ppm. A cold rolled CpTi substrate served as the build plate for the process. The DED system utilizes coaxially flown premixed powders from the laser head, converging on a point where the laser melts the powders on the build plate. The specific print parameters are provided in Table 1. Samples intended for in vivo study were printed on an SLM-based powder bed fusion (PBF) system, 3D Systems ProX® DMP 200 (3D Systems, Rock Hill, SC). This system is equipped with a 300 W fiber laser having a wavelength of 2=1070 nm. Porous structures with a diameter of 2.4 mm and a height of 4 mm, comprising ˜40 vol. % porosity, were designed using 3DXpert CAD Software (3D Systems in Rock Hill, SC). The premixed powders were placed in the supply chamber and compacted using a compaction plate. A thick CpTi plate with a thickness of approximately 2.5 cm was utilized as the build platform and securely positioned on the build stage. A roller system transported the powders from the supply to the build stage, with a layer thickness of 30 μm. The laser power and scanning speed for all the compositions are reported in Table 7. After printing, porous cylinders were cut from the build plate and subjected to repeated sonication in de-ionized (DI) water and ethanol, followed by compressed air treatment to remove loose powder particles from the pores. The final step involved acid etching in 1% HF in DI water to remove loosely attached powders. The samples were then sonicated again in DI water and ethanol to remove any acid residues.

TABLE 7
Additive manufacturing of CpTi, CpTi—SiO₂, and CpTi—SiO₂-3Cu
compositions via DED and PBF operations.

| | DED (in vitro, microstructure, hardness testing samples) | | | | | | | |
|---|---|---|---|---|---|---|---|---|
| | Laser | | Scan speed | | | | | |
| | power (W) | | (mm/min) | | Shield gas | Carrier gas | Powder disc | Slice |
| Composition | Contour | Hatch | Contour | Hatch | (l/min) | (l/min) | speed (rpm) | (mm) |
| CpTi | 350 | 350 | 1500 | 1200 | 18 | 14 | 0.7 | 0.3 |
| CpTi—SiO2 | | | | | | | | |
| CpTi—SiO2-3Cu | | | | | | | | |

PBF (in vivo samples with 40% porosity)

| | Laser | Scan | |
|---|---|---|---|
| | power | speed | Slice |
| Composition | (W) | (mm/s) | (μm) |
| CpTi | 180 | 1600 | 30 |
| CpTi—SiO2 | | | |
| CpTi—SiO2-3Cu | 198 | 1440 | |

### Phase Analysis, Microhardness, and Microstructure

DED-printed discs were cut from the build plate into individual discs using a water-jet system and subjected to grinding using SiC grinding papers with grit sizes ranging from 80 to 1200. Subsequently, alumina suspension polishing was done with a stepwise reduction of particle size of the alumina powder suspended in DI water from 1 to 0.05 μm. The polished discs were then analyzed for phase detection, microhardness, and microstructure. Phase analysis was done on a Siemens D5000 x-ray diffraction system (Siemens, Washington DC, USA) equipped with a θ-θ goniometer geometry using a Kα radiation of 15.4 nm wavelength. The intensity of the peaks within the 30°≤2θ≤80° range was observed. Microhardness testing was performed using a Phase II Plus Micro Vickers Hardness tester (Upper Saddle River, NJ, USA) with a 200 g load and a dwell time of 15 s. For microstructural analysis, the polished surfaces of the discs were etched for 45 s using Kroll's reagent and examined on a Keyance VHX-970FN optical microscope (Keyance Corporation of America, Itasca, IL).

In Vitro Bacterial Study-Staphylococcus aureus Bacterial culture was performed on CpTi (negative control) and CpTi—SiO₂-3Cu samples to assess the antibacterial resistance against the gram-positive bacterial strain Staphylococcus aureus. The culture was conducted for 24, 48, and 72 h. Freeze-dried S. aureus (Carolina Biological, NC) was rehydrated using a rehydration media. The rehydrated bacteria were serial diluted in nutrient broth to achieve an optical density of 0.5 McFarland standard, corresponding to 1.5×108 CFU/ml of bacteria. Before the culture, the polished disc samples were sterilized and placed in 24-well plates. The experiments were conducted in triplicate for agar plate colony count and duplicates for SEM characterization. 106 CFU of bacterial colonies were seeded on the surface of the discs with 2 ml of tryptic soy broth added to each well as the nutrient medium. Agar plate characterization was employed for 24 h, and SEM for all three-time points. For agar plate characterization, bacterial cells from the triplicate samples for agar plate count were scraped using cell scrapers and mixed with 2 ml of 0.1 M PBS. The mixture was then serially diluted to yield a solution containing approximately 10 to 100 colonies in 10 μl of the PBS solution. 10 μl volume of this solution was streaked on a tryptic soy agar plate and incubated for 24 h. The antibacterial efficacy was evaluated by comparing the bacterial colonies counted on the different material compositions. The calculation of colony count (N) was determined using the formula;

$$N = C \times d \times 1000 / l \qquad ( 1 )$$

where C represents the average colony count on a plate, d is the dilution factor, and l is the volume of bacterial suspension on the sample. The antibacterial resistance (R) was expressed as a percentage using the equation,

$$R = ( {N}_{control} - {N}_{material} ) / {N}_{control} \times 100 \% , \qquad ( 2 )$$

The duplicate samples for observing bacterial cell morphology were preserved in a fixative solution of 2% glutaraldehyde and 2% paraformaldehyde in 0.1 phosphate buffer overnight at 4° C. The samples were thrice rinsed in 0.1 M PBS and subjected to dehydration in 2% osmium tetraoxide (OsO4) for 2 h. The samples were then subjected to serial dehydration in ethanol and finally critical drying using hexadimethylsilazane (HMDS). These samples were examined under a scanning electron microscope (SEM) after gold coating on Apreo, Thermo Scientific, MA, USA. Bacterial efficiency at the end of each 24-48-72 h was calculated from bacterial colony counts from triplicate SEM images captured at 5000× magnification using equation (2).

### In Vivo Study

CpTi, CpTi—SiO₂, and CpTi—SiO₂-3Cu compositions were subjected to an in vivo rat study. Adding SiO₂ to CpTi is expected to show better early-stage osseointegration performance than CpTi. PBF-fabricated porous implants with ˜40 vol. % porosity were used for the study.

### Surgery and Implantation Procedure

For the in vivo study, male Sprague-Dawley rats weighing 300-350 grams were used. Upon procurement, the rats were acclimatized in separate cages within temperature and humidity-controlled rooms for at least two weeks. 30 min before the surgery, the rats were subcutaneously administered Buprenorphine (0.3 mg/kg) to alleviate the pain. Anesthesia was induced using a prescribed dose of IsoFlo® (Isoflurane, USP, Abbott Laboratories, North Chicago, IL, USA) in combination with oxygen. Once the rats were fully anesthetized and immobile, the surgical area around the femur and knee was shaved and cleaned meticulously with alternate chlorhexidine and isopropyl alcohol scrubs. 0.3 ml of lidocaine HCL (without epinephrine), 0.5% was injected subcutaneously near the implantation area on each leg to provide localized numbing. The rats under anesthesia were then placed on a sterile surgical table. A 2-inch incision was made along the lateral side of the femur, above the distal femoral condyle. Using gradually increasing drill bits, a unicortical defect with a diameter of 2.4 mm was created on the lateral epicondyle. Saline was used to rinse the area during the drilling process to prevent thermal necrosis and remove bone fragments. The implant was inserted, and the fascia over the incision site, followed by the skin, was sutured using undyed braided coated MONOCRYL-polyglactin 910 sutures (Ethicon Inc., Somerville, NJ, USA). The incision area was cleaned with saline scrubs and stapled. The same procedure was performed on the other leg of each rat. Throughout the surgery, the rats' respiration rate was monitored periodically. After the surgery, the rats were administered subcutaneous injections of lactated ringers' solution (LRS, 3 ml) for rehydration, followed by meloxicam (0.2 mg/kg) as an anti-inflammatory analgesic. The rats were closely monitored until they regained consciousness. Postoperative care was provided for 3 days, including buprenorphine administration every 12 hours and meloxicam administration every 24 h. After 6 weeks, the rats were euthanized using carbon dioxide overdose, followed by cervical dislocation as a secondary measure. The femoral bone with the metal implant was then harvested. These bone-metal explants were fixed in 10% neutral buffered formalin for a minimum of 72 h to facilitate tissue infiltration. The experimental and surgical procedures followed the approved protocol of the Institutional Animal Care and Use Committee (IACUC) at Washington State University (Pullman, WA).

### Histological and Histomorphometric Analysis

The bone-metal explants were immersed in 10% neutral buffered formalin for at least 72 hours to facilitate tissue infiltration. Subsequently, a series of dehydration steps in ethanol was performed, followed by embedding the specimens in polymethyl methacrylate (PMMA). These embedded bone explants were sliced into thin sections measuring 200 μm using an Ekakt™ saw. The sections were then mounted on glass slides and further reduced to 20-50 μm thickness using 1200 grit-size sanding paper on an Ekakt 400 micro grinder. Once the desired thickness was achieved, the sections were polished using 4000 grit-size paper on the micro grinder. To analyze the bone sections, Gomori trichrome, Hematoxylin & Eosin (H&E), and Sanderson's Rapid Bone Staining (SRBS) were applied as different staining techniques for each composition. The stained bone sections were observed using a Keyance digital microscope (Model VHX-7000, Itasca, IL). Visible markers indicating an inflammatory response in the vicinity of the implant were examined through imaging of slides stained with H&E. Muscle fibers and the presence of collagen at the bone-implant contact (BIC) were observed on Gomori trichrome-stained slides. Mineralized bone formation, the presence of osteoid at the bone-implant contact (BIC), and mineralization fronts were assessed on SRBS-stained slides. Histomorphometric analysis was conducted using SRBS-stained slides for each composition. To focus on the region of interest (ROI) within 100-150 μm from the implant surface, images were captured at 1000× magnification around the BIC area for the SRBS-stained slides. Quantitative evaluation of mineralized bone formation at the BIC was performed using Trainable Weka Segmentation in ImageJ with the Random Forest Algorithm applied to individual images. A minimum of 7 regions were analyzed to quantify the extent of mineralized bone formation at the BIC, which was presented as the percentage of area fraction.

### Results

CpTi is used for surface coating on Ti6Al4V at load-bearing hip, knee, ankle, and spinal devices or as the sole implant material at low load-bearing sites, such as maxillofacial and dental implants. This study introduced enhanced biological functionalities in the CpTi matrix by SiO₂ and Cu addition. Physical and mechanical characterizations have been carried out via microstructure, Vickers microhardness, and phase analysis. The biological performance of the CpTi—SiO₂-3Cu chemical makeup has been evaluated by studying the bone-formation characteristics in vivo using a rat distal femur model. Antibacterial efficacy was measured via in vitro bacterial culture using the most commonly occurring bacterial infection strain, Staphylococcus aureus or S. aureus.

### Physical and Mechanical Properties

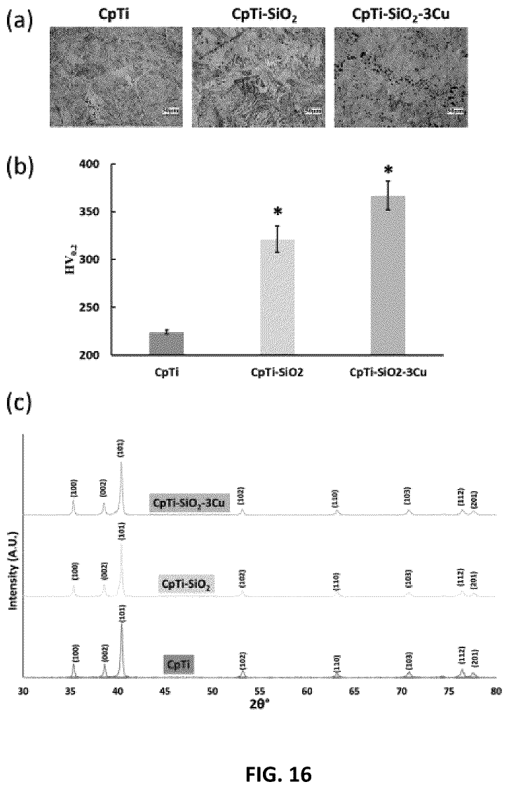

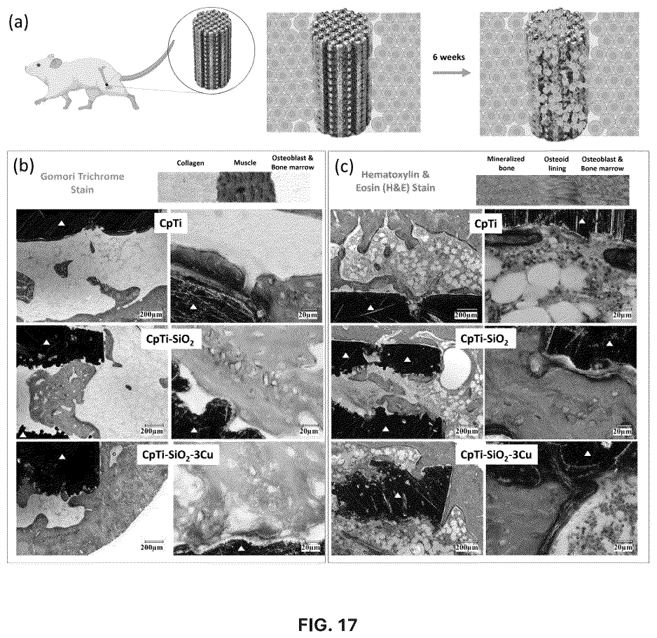

SLM process involves a high cooling rate (˜104-106 K/s) that prevents long-range diffusion and thermodynamically stable phase formation, α(hcp) phase in the case of CpTi. Non-equilibrium martensitic phases are formed featuring metastable α′—Ti phases with acicular needle-like morphology, FIG. 16a. This martensitic α′—Ti enhances the microhardness of SLM-processed CpTi over cold-rolled CpTi; the microhardness of CpTi was observed to be 224±2 HV0.2. With SiO₂ addition in CpTi, CpTi—SiO₂ resulted in a similar acicular α′—Ti needle-like microstructure. The hardness is increased with 1 wt. % SiO₂ ceramic introduction in the CpTi matrix, 321±12 HV0.2 for CpTi—SiO₂ (FIG. 16b). Cu addition in CpTi—SiO₂ shows keyhole porosities on the surface of CpTi—SiO₂-3Cu, FIG. 16a. Cu has a very high thermal diffusivity (˜100× to that of CpTi), low laser absorption (98% reflectivity), and high viscosity in molten state (1.96 mPa-s). Laser melting of Cu creates a wider and shallower melt pool with molten particles splashing, leading to keyhole porosities. CpTi—SiO₂-3Cu exhibits the highest hardness value of 367±15 HV0.2 due to Ti2Cu intermetallic phase formation and solid-solution strengthening mechanism by Cu solute particles. Although the effect of composition was observed in microstructure and microhardness, phase analysis did not reveal any significant differences among the compositions. All peaks observed were that of α′—Ti phase, as shown in FIG. 16c, typically observed in AM-processed CpTi. Similarly, the XRD patterns of CpTi—SiO₂ and CpTi—SiO₂-3Cu exhibited similar peaks to CpTi due to low amounts of SiO₂ and Cu addition in CpTi. No individual peaks for Cu or SiO₂ presence were observed, suggesting homogeneous compositions post-AM operation.

### Histological Analysis and Histomorphometry of Bone-Implant Interface

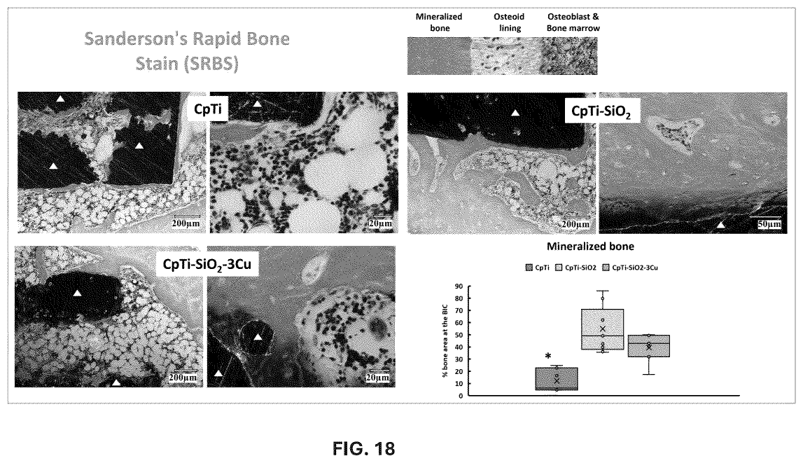

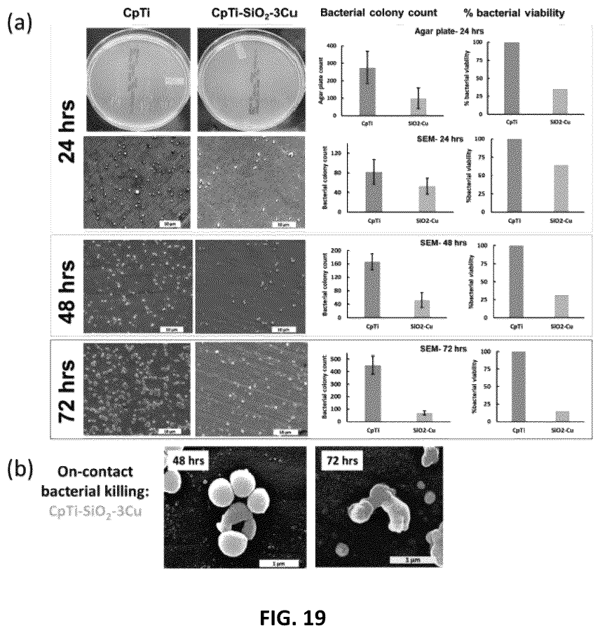

Assessing the efficacy of the material composition in vivo is essential for a comprehensive understanding of its biological performance. A successful biological fixation of an implant is governed by bone-implant contact (BIC), a critical indicator of osseointegration. CpTi, CpTi-SiO₂, and CpTi—SiO₂-3Cu were subjected to an in vivo rat distal femur model, with CpTi as the control. The bone sections post-explantation were subjected to various staining procedures such as Gomori Trichrome, Hematoxylin & Eosin (H&E), and Sanderson's Rapid Bone Staining (SRBS), FIG. 17 and FIG. 18, respectively, each indicating distinct colors representing specific bone forming features at the BIC. H&E-stained bone sections were assessed for signs of inflammation caused by cytotoxicity; all compositions showed no signs of inflammatory response, including neoplasia and necrosis. Gomori Trichrome stain, FIG. 19b, shows shades of blue and pink, representing collagen formation and muscle fiber growth at the BIC. All compositions display collagen formation with interwoven muscle fibers at the BIC as the mineralization fronts. CpTi visibly shows gaps at the BIC while CpTi—SiO₂ and CpTi—SiO₂-3Cu show mineralization front apposed to the implant outer surface, indicating better osseointegration. These mineralization fronts can be observed infiltrating the implant surface for CpTi—SiO₂ and CpTi—SiO₂-3Cu, resulting in an uneven boundary at the BIC. This infiltrative nature of mineralization fronts in CpTi—SiO₂ and CpTi—SiO₂-3Cu indicates a higher affinity of new bone formation towards the CpTi—SiO₂-3Cu material chemistry, distinguishing them from CpTi, where a clear boundary between the implant outer surface and the bone region is seen. The observations from H&E-stained slides support higher osseointegration performance exhibited by CpTi—SiO₂ and CpTi—SiO₂-3Cu compared to CpTi, FIG. 19c. The varying shades of pink in the H&E-stained histology indicate distinct features such as osteoid lining fronts at the BIC trabecular bone formation and osteoblast and bone marrow regions. The bone formation process at the BIC follows osteoblastic recruitment, leading to osteoid tissue and eventual maturation into mineralized bone. An osteoid lining can be observed at the BIC for CpTi, followed by trabecular bone in certain areas and absence in the rest replaced by osteoblastic regions. In contrast, CpTi—SiO₂ displays an extended region of this mineralized bone formation at the BIC, with bone apposition to the implant surface in certain regions. Upon closer examination, the mineralization fronts can be observed to infiltrate the implant region for CpTi-SiO₂ composition. This indicates enhanced early-stage osseointegration for CpTi—SiO₂ than CpTi attributed to angiogenic properties of SiO₂. Similar characteristics are observed for CpTi-SiO₂-3Cu with higher bone formation at the BIC and tissue infiltration in the implant region on its outer surface. Porosity aids in enhanced osseointegration capabilities. Infiltration inside the pores can be observed for CpTi—SiO₂ with bone-rich regions apposed to the implant surface. Although CpTi—SiO₂-3Cu exhibits good tissue infiltration inside the pores, osteoblast and bone marrow-rich regions at the implant surface can be observed with lower bone formation. CpTi-SiO₂-3Cu, thus, showed a delayed osseointegration performance in comparison to CpTi—SiO₂.

In contrast to H&E-stained histology, SRBS-stained micrographs (FIG. 18) show a better contrast of colors for a better understanding of the implant and host bone interactions and bone remodeling. CpTi exhibits an osteoid lining followed by trabecular bone in some regions and absence in others, consistent with the observations from the H&E-stained histology. CpTi—SiO₂ shows a thicker region of bone formation at the implant's outer surface. Possible gaps at the BIC are filled with mineralization fronts of osteoid tissue, while gaps at the interface were observed for CpTi from Gomori trichrome-stained bone sections, FIG. 18. SRBS-stained micrographs for CpTi—SiO₂-3Cu also show mineralized bone formation at the BIC; higher magnification images reveal encompassing of implant region by mineralized bone formation. Quantitative histomorphometric analysis was implemented to quantify the trabecular bone formation at the BIC within a region of interest of 100 μm from the implant's outer surface. With 54.8±17.8%, CpTi—SiO₂ showed the highest bone formation at the BIC, revealing enhanced osseointegration and remodeling. CpTi—SiO₂-3Cu showed a lower quantity of bone formed at the BIC of 39.4±10.6%. The values for CpTi—SiO₂ and CpTi—SiO₂-3Cu fall within the error range of each other and are statistically similar; the difference in mean values can be attributed to delayed osseointegration in CpTi—SiO₂-3Cu due to the presence of Cu. CpTi displayed the poorest bone remodeling capabilities among the compositions, with minimum trabecular bone formation at the BIC, measuring 12.1±8.4%. Histology and histomorphometric evaluations reveal higher affinity of new tissue formation from host bone and enhanced early-stage osseointegration performance by CpTi—SiO₂ and CpTi—SiO₂-3Cu chemical makeup compared to CpTi due to the presence of SiO₂.

### Infection Prevention Capability

Bactericidal performance is one of the most essential sought-after properties in an implant material. With the widespread prevalence of postoperative bacterial infections at the implant site, an implant material with antibacterial capabilities will minimize the need for revision surgical procedures and implant replacement, ensuring implant longevity in vivo. Since CpTi does not possess inherent antibacterial capabilities, this study added bactericidal Cu to CpTi to incorporate antibacterial performance via on-contact bacterial killing exhibited by Cu. Bacterial viability for CpTi—SiO₂-3Cu was tested against CpTi control with gram-positive S. aureus bacterial stain. FIG. 19a reports agar plate (24 h) and SEM images (24-48-72 h) on the surface of CpTi and CpTi—SiO₂-3Cu with their respective bacterial colony counts and % bacterial viability. The agar plate count at the end of 24 h timepoint revealed 64% antibacterial efficacy by CpTi—SiO₂-3Cu to CpTi. SEM counting of bacterial cells at 24 h timepoint reveals a similar antibacterial efficacy of 57%. At the end of 48 h, CpTi—SiO₂-3Cu exhibited a similar antibacterial efficacy of 53%. The highest antibacterial performance by CpTi—SiO₂-3Cu was observed at the end of 72 h, with 85% reduction in planktonic bacteria on its surface. The antibacterial performance of CpTi—SiO₂-3Cu composition is observed to increase from 24 to 72 h. FIG. 19b shows evidence of Cu's on-contact killing nature of S. aureus bacterium on the surface of CpTi—SiO₂-3Cu post 48 and 72 h. Rupture of bacterial cell membrane walls can be observed at the end of 48 h timepoint. 72 h image shows bacterial cells adhering to each other, causing septum formation and eventual cytoplasm outflow by cell membrane disruption. CpTi-SiO₂-3Cu exhibited excellent antibacterial resistance, with an eventual increase in bacterial growth inhibition and bactericidal performance from 24 h (57%) to 72 h (85%). The longevity of an implant in vivo is governed by its ability to achieve accelerated early-stage osseointegration and prevent polymicrobial infections at the implant site. The current choice of metallic material, Ti, does not offer either of these desired characteristics. To address these limitations, current strategies involve a surface modification of Ti with antibacterial coatings and bioactive ceramics with osteogenic and angiogenic dopants. The long-term stability is still compromised either due to desorption of the antibacterial and osteogenic agents from the implant surface with time or cement debonding and delamination, causing implant failures. Alarmingly high failure rates have been observed in implants due to these issues: 20-47% in dental implants due to peri-implantitis infections and 6% (2009-2017) in tibial implants due to aseptic loosening in cemented implants.

Ti is biocompatible yet bioinert. It does not aid in accelerated bone healing and attachment to host bone, an essential factor for patients with compromised bone health. In this study, SiO₂ was added as an angiogenic agent for accelerated tissue engineering. Si4+ ions are present in trace amounts in the inorganic bone and are known to stimulate angiogenesis, i.e., help generate new blood vessels sprouting from existing ones (host bone). This angiogenic activity is essential in the regeneration and repair of bone and muscle on the implant surface since blood vessels supply essential nutrients and oxygen. The bone regeneration process starts with stimulating stem cells to enable angiogenesis, followed by osteogenic stimulation toward vascular bone formation. Si4+ ion incorporation in bioglass, crystalline ceramics, and composites such as calcium phosphate and hydroxyapatite have been found to promote osteogenesis and angiogenesis. The material makeup with ceramic matrix does not qualify for a long-term implant application due to its bioresorbable nature. The brittleness of the ceramic matrix is another concern with the chances of implant failure. However, SiO₂ in CpTi matrix investigated in this study can be used as a potential long-term implant surface composition. From histology images for CpTi—SiO₂, the tissue growth on the implant's outer surface can be seen to infiltrate the implant region, suggesting a higher affinity of tissue growth to the material composition. On the contrary, CpTi showed gaps between the mineralization front and the implant's outer surface, revealing slower osseointegration. Quantitative histomorphometry from SRBS-stained histology micrographs supports the qualitative results; with 4.5× higher mineralized bone formation at the bone-implant interface than in CpTi, CpTi—SiO₂ exhibited enhanced osteogenic performance.

Curbing implant-related infections is another essential parameter contributing to the implant's long-term longevity and stability. Secondary infections at the implant site necessitate revision surgery procedures, further deteriorating the patient's health. Cu is a popular antibacterial agent alloyed with Ti. Cu is a necessary trace element in the body, but higher amounts of Cu are cytotoxic. With studies on varying amounts of Cu in Ti, a debate persists on the optimum amount of Cu in Ti without causing cytotoxicity. In this study, 3 wt. % Cu was added to the CpTi-matrix with 1 wt. % SiO₂ for osteogenesis and angiogenesis. From the H&E histology micrographs, FIG. 19c, no inflammatory markers were observed, suggesting CpTi—SiO₂-3Cu composition is non-toxic. However, a delayed osseointegration performance was observed with Cu addition to CpTi—SiO₂; quantitative histomorphometry from SRBS-stained histology revealed a lower amount of mineralized bone formation at the bone-implant contact, though statistically similar to that for CpTi—SiO₂. Despite the delayed osseointegration, the mineralized bone formation in CpTi—SiO₂-3Cu was 3 times higher than in CpTi due to the presence of SiO₂. The antibacterial efficacy of Cu in CpTi—SiO₂-3Cu to non-antibacterial CpTi increased gradually with time, with the highest efficacy of 85% at the end of 72 h.

CpTi—SiO₂-3Cu composition showed no cytotoxicity in vivo with excellent antibacterial capabilities and superior osteogenic performance to CpTi. This composition displayed enhanced biological performance towards the two primary parameters responsible for implant longevity and stability: enhanced early-stage osseointegration and bactericidal performance. CpTi—SiO₂-3Cu composition has the potential to replace CpTi as the material of choice for low load-bearing implant sites such as dental and cranial devices and coating on Ti6Al4V for high load-bearing implants such as hip and knee prosthesis.

The implant's in vivo longevity and stability is primarily governed by its early-stage attachment to the host bone. Aseptic loosening of implants, either due to improper bone attachment or infections, necessitates revision procedures that affect the patient's health. CpTi does not offer either enhanced early-stage osseointegration or antibacterial performance. Despite this, CpTi is still used as the preferred implant material for low load-bearing applications or as a porous coating on Ti6Al4V at load-bearing sites. To overcome the shortcomings of CpTi, we have explored a multifaceted CpTi chemical makeup: 1 wt. % angiogenic-osteogenic SiO₂ and 3 wt. % Cu added to CpTi. These compositions were processed via additive manufacturing (AM) since it provides freedom of design; AM enables processing structures with designed porosities, promoting osseointegration at the implant's surface. In vivo studies revealed superior osseointegration performance by CpTi—SiO₂ composition over CpTi. The qualitative comparison revealed gaps at the bone-implant interface for CpTi, whereas tissue infiltration into the implant's outer surface for CpTi—SiO₂ suggests a higher affinity of tissue growth and maturation towards the CpTi—SiO₂ chemical makeup. Quantitative histomorphometry revealed 4.5 times higher bone formation in CpTi—SiO₂ than in CpTi. Although Cu is cytotoxic in large amounts, 3 wt. % of Cu in CpTi—SiO₂-3Cu showed no inflammatory markers but slightly delayed osseointegration compared to CpTi—SiO₂. CpTi—SiO₂-3Cu displayed 3 times more bone formation than CpTi. In vitro bacterial studies with S. aureus revealed 85% antibacterial efficiency for CpTi—SiO₂-3Cu to CpTi at the end of 72 h. With enhanced bone formation and maturation and excellent antibacterial capability displayed, CpTi—SiO₂-3Cu with enhanced biological functionalities has the potential to replace CpTi, ensuring a reduction in revision surgery needs owing to poor implant attachment and bacterial infections.

In view of the many possible embodiments to which the principles of the disclosed compositions and methods may be applied, it should be recognized that the illustrated embodiments are only preferred examples and should not be taken as limiting the scope of the disclosed compositions and methods.

What is claimed is:

1. A biocompatible alloy comprising:
titanium, copper, and
at least one metallic element selected from: tantalum, niobium, zirconium, zinc, tungsten, lithium, potassium, strontium, sodium, calcium, chromium, molybdenum, tin, manganese, iron, and cobalt.

2. The biocompatible alloy of claim 1, wherein the at least one metallic element is present in an amount up to 50 wt %.

3. The biocompatible alloy of claim 1, wherein the biocompatible metal alloy is configured as a medical implant shaped for a subject body part.

4. The biocompatible metal alloy of claim 1, wherein the biocompatible metal alloy is configured as an industrial component.

5. The biocompatible alloy of claim 1, further comprising aluminum and vanadium.

6. The biocompatible alloy of claim 1, further comprising zinc.

7. A biocompatible alloy comprising (i) titanium, (ii) copper and (iii) tantalum, niobium, or a mixture thereof.

8. The biocompatible alloy of claim 7, wherein the biocompatible alloy comprises (i) titanium, (ii) copper and (iii) tantalum.

9. The biocompatible alloy of claim 7, wherein the biocompatible alloy comprises (i) titanium, (ii) copper and (iii) niobium.

10. The biocompatible alloy of claim 7, wherein the biocompatible alloy comprises titanium, copper, tantalum, and niobium.

11. The biocompatible alloy of claim 8 wherein the tantalum is present in an amount of up to about 50 wt %.

12. The biocompatible alloy of claim 9, wherein the niobium is present in an amount of up to about 50 wt %.

13. The biocompatible alloy of claim 7, wherein the copper is present in an amount of about 0.01 wt % to about 30 wt %.

14. The biocompatible alloy of claim 11, wherein the copper is present in an amount of about 0.01 wt % to about 30 wt %.

15. The biocompatible alloy of claim 12, wherein the copper is present in an amount of about 0.01 wt % to about 30 wt %.

16. The biocompatible alloy of claim 7, further comprising aluminum and vanadium.

17. The biocompatible alloy of claim 7, further comprising zinc.

18. The biocompatible alloy of claim 7, wherein the biocompatible metal alloy is configured as a medical implant shaped for a subject body part.

19. The biocompatible metal alloy of claim 7, wherein the biocompatible metal alloy is configured as an industrial component.

20. A biocompatible alloy comprising (i) titanium, (ii) copper, and (iii) magnesium oxide, silicon dioxide, or a mixture thereof.
